(19) United States
(12) Patent Application Publication
(10) Pub. No.: US 2003/0099747 A1
(43) Pub. Date: May 29, 2003

(54) Thickened oil compositions of edible oil

(76) Inventors: Meir Eini, Nes Ziona (IL); Dov Tamarkin, Maccabim (IL)

Correspondence Address: Anthony Castorina, G E Ehrlich, Suite 207, 2001 Jefferson Davis Highway, Arlington, VA, 22202, US

(21) Appl. No.: 10/169,804

(22) PCT Filed: Jan. 10, 2001
(86) PCT No.: PCT/IL01/00024

(30) Foreign Application Priority Data
Jan. 10, 2000 (IL) 133968
Jan. 10, 2000 (IL) 133969
Jun. 27, 2000 (IL) 137051
Jun. 27, 2000 (IL) 137052

(51) Int. Cl. A23B 4/00

(52) U.S. Cl. 426/401

(57) ABSTRACT

Thickened, preferably thixotropic oil compositions are disclosed comprised of high proportions of edible oils, in particular unsaturated oils, and edible solidifying agents, in particular long chain fatty acids and/or long chain fatty alcohols, for use in the preparation of edible foods and medicinal/therapeutic products. Further disclosed are methods for the production of the thickened, thixotropic oil compositions, edible food and medicinal/therapeutic products containing same and methods of their production and consumption.

### FIELD AND BACKGROUND OF THE INVENTION

The present invention relates to a thickened oil composition of edible oil and the manufacture of edible foods, food supplements and medicinal products therefrom. More particularly, the present invention relates to (i) a thickened and/or thixotropic composition of an edible unsaturated oil, characterized by both semi-solid and liquid form, depending on temperature and/or applied mechanical (shear) forces; (ii) methods of preparing same; and (iii) methods of preparing edible foods, food supplements and medicinal products therefrom.

Oily substances are well known in the art and are used in the manufacture of food and medicinal/therapeutic products. The wide use of oily substances in food and medicine manufacturing is attributed to various properties of such substances, amongst which are the health benefits and hydrophobicity thereof, and their ability to dissolve oil-soluble constituents with either nutritional and/or therapeutic value.

Of great importance are oily substances that comprise unsaturated or poly-unsaturated fatty acids, also defined herein as unsaturated oils. Due to their molecular structure which comprise cis-double bonds, unsaturated oils are known to exert health benefits exceeding those exerted by saturated oils. However, the unsaturated nature such fatty acids is further responsible for their being liquid at room temperature, which is contrary to the highly advantageous solid or semi-solid state of oily substances in the manufacture of food and medicinal/therapeutic products. Therefore, immense efforts are made to produce solidified unsaturated oils.

The common process used to solidify unsaturated oils consists of partial or full hydrogenation of such oils, so as to obtain semi-solid oils. Yet, because of this chemical transformation, the oils become saturated and lose their healthy properties. It is well known in the art, and was further stated by the Food and Drug Administration (FDA), that the intake of saturated fat is linked to high blood cholesterol, which in turn is linked to increased risk of coronary heart disease (See “Lowering Cholesterol” in FDA Consumer, March 1994, and “A Consumer's Guide to Fats” in FDA Consumer, May 1994). The partial hydrogenation process also results in the formation of “trans”-fatty acids, which have recently been shown to possess a plurality of adverse properties. Structurally, trans-fatty acids are similar to saturated fatty acids and hence they influence cell membranes in the same way. Saturated fatty acids elevate the LDL cholesterol levels by inhibiting the LDL receptors and thus inhibiting the removal of cholesterol from the blood. Trans-fatty acids can also raise LDL cholesterol levels in the blood. Trans-fatty acids apparently elevate the level of lipoprotein[a], a risk factor for heart disease, as well. Furthermore, at high levels of intake (3-4 times the normal US consumption levels), but not at levels that are currently consumed, trans-fatty acids can modestly reduce the blood levels of HDL cholesterol (the good cholesterol) and also interfere with the metabolism of the essential fatty acids. In the Nurses Health Study in Boston it was reported that intake of trans-fatty acids was associated with an increased risk of coronary heart disease. Another example relates to trans-linoleates, which devoid of essential fatty acid activity. Animal studies have shown that trans-fatty acids retard growth more than diets deficient in essential fatty acids. Trans, trans-18:2-α6-linoleic acid decreases the conversion of linoleic acid to gamma-linolenic acid in the liver microsomes (Handbook of Lipids in Human Nutrition. Gene Spiller, Ed. (1996) page 92). Trans-fatty acids can enter fetus and breast milk. They can be incorporated into a child's developing brain instead of more beneficial fatty acids. A recent European epidemiological study has also revealed an association between ingestion of sizable amounts of trans-fatty acids and the prevalence of asthma, allergic rhino conjunctivitis and atopic dermatitis (Lancet 1999; 353:2040-41). The Food and Drug Administration (FDA) has recently stated that the consumption of saturated oils, which contain “trans”-fatty acids contributes as well to increased blood LDL-cholesterol (“bad” cholesterol) levels, which increase the risk of coronary heart disease (see, U.S. Department of Health and Human Services, Press Release, Nov. 12, 1999). Hence, by solidifying unsaturated oils by an hydrogenation process, the beneficial properties of the unsaturated oils are substituted by the highly undesirable adverse properties of the saturated oils and the formation of “trans”-fatty acids.

Reference is now made to several aspects in which the aforementioned advantages of solid or semi-solid unsaturated oils with respect to food and medicinal/therapeutic products are demonstrated:

Unsaturated Oils for Medical/Therapeutic Use:

Natural oils of vegetable and marine origin are known for their health benefits, which is attributed to the unsaturated and poly-unsaturated fatty acids included in the triglyceride components thereof. Furthermore, such natural oils contain essential nutritional constituents, such as oil-soluble vitamins (e.g., vitamin A and vitamin E), minerals and other therapeutically beneficial constituents.

Another class of oils having a therapeutic value includes mineral and silicon oils which are known to be useful for the treatment of constipation and other medical disorders, and are, similar to the natural oils derived from marine animals and vegetables, liquid at ambient temperature.

However, the administration of such therapeutically beneficiary oils is limited by their liquid form since they cannot be ingested by consumes in amounts sufficient to exert therapeutic affects, due to the unpleasant taste, and/or texture thereof.

Furthermore, the commonly used soft gel capsules, which hinder the taste and oily sensation of the oils, do not provide satisfactory alternative, since they can contain limited amounts of oil, in the range of 0.2 to 0.5 grams, which is, in many cases, far less than the daily-recommended therapeutic amount, (ranging from 1 to 20 grams per day). Swallowing many such capsules per day on a continuous basis is not practical. Larger capsules (up to 1.5 ml) are known, but they are difficult to swallow.

Thus, converting liquid therapeutic oils into semi-solids could conceivably provide a favorable way to enable ingestion of therapeutic amounts of such oils.

Unsaturated Oils as Food Constituents or Coatings:

Oily substances are commonly used in the manufacturing of food products in order to protect food from moisture as, e.g., in the case of pastry and meat, or to protect it from loosing moisture as, e.g., in the case of popcorn kernels. Even application of the oil is highly important with respect to these objects. Moreover, oily substances can further serve as a delivery system of flavoring and therapeutic agents onto the food.

The oil must be in a solid phase in order to stick to the food. It must also be solid in order to facilitate even delivery and distribution of flavoring and therapeutic additives onto the food product. Since the use of naturally liquid unsaturated oils for food production is highly advantageous due to their health benefits, a solidifying process of these oils is required. However, as is discussed hereinabove, this process involves a chemical modification of the oil which takes away the beneficial properties of unsaturated oils.

Thus, it would be desirable to provide a composition for applying coating, flavoring additives and/or therapeutic additives to foods, which is easy to prepare, evenly distributable, but does not contain saturated fats. It would be particularly desirable to provide such a composition for applying flavoring agents to foods.

It would be further desirable to provide a method for making flavored food, which is easy to prepare, has an even flavor distribution, but does not coils.

It would be further desirable if this method for applying flavoring was applicable to foods, such as popcorn, pastry, vegetables, potatoes, pasta, rice, fish or meats.

Especially desirable is a composition and method for applying oil and optionally flavoring components to popcorn for mass production and microwave popping systems. Popcorn is a food product which requires very careful handling in order to ensure large tender kernels when popped. It is a well-known fact that unless precautions are taken to preserve the moisture in the raw corn, it soon dries out, after which no popping method produces a satisfactory product. Oil coating is known to preserve moisture, and obviously, an even distribution of the oil is essential to protect the whole mass of the corn. It is also accepted that the proportion between oil and corn affects the results obtained. Too little oil results in small kernels, whereas too much results in soggy corn with inferior smell and taste.

The most common sources of oil for popping popcorn are coconut oil or liquid vegetable oils, which are characterized by a high degree of unsaturation, e.g., corn oil, soybean oil, and certain partially hydrogenated edible oils like cottonseed.

Trends in the state of the art in the popping of corn have been to make the total operation simpler. This has involved attempts to put together in a single package more than one of the ingredients. For instance, U.S. Pat. No. 2,604,407 teaches that it is possible to preserve moisture in popcorn kernels by coating the kernels with the correct amount of liquid fat (vegetable oil) in such manner that homogeneous parcels with the correct amounts of corn and oil may be obtained for individual use. However, this method cannot ensure avoidance of oil spillage. U.S. Pat. No. 2,518,247 teaches the use of a total combined package of popcorn, oil (used as binder), salt, color, and flavoring.

Many attempts have been made in order to improve the even distribution of oil-flavor systems on corn. These attempts commonly comprise the use of semi-solid oils, obtained by partial or full hydrogenation of unsaturated oils (examples are found in U.S. Pat. Nos. 4,163,066 and 4,888,186). Yet, because of the hydrogenation process, the oils become saturated and lose their healthy properties. Thus, it would be desirable to have an oil-flavor composition that comprise semi-solid oils with high degree of unsaturation

Unsaturated Fats as Modified Fat Substitutes:

Edible fat-based materials, such as shortening, margarine, butter, and cooking oils are an essential ingredient in the preparation of a wide variety of foods. Fats and oils are used in conventional food processing techniques such as cooking, frying, and baking and are also employed as ingredients to bestow desirable properties of taste, texture, form, and stability to food products.

Among the physicochemical properties of fats and oils, which are critical to their utility as food ingredients, are viscosity and melting temperature. Many of the common fat-based edible materials, including those comprising saturated, mono-unsaturated, and poly-unsaturated oils, maintain a liquid or semi-solid state at ambient temperatures of traditional food storage and consumption conditions, and thus many food products suffer from instability towards the typical environmental conditions to which they are exposed. It would thus be desirable to have more stable food products as well as methods for their production based upon the use of edible fat-based materials modified to a higher degree of viscosity and melting temperature. In order to obtain more solid consistency, more highly hydrogenated oils characterized also by trans fats are often employed in food processing, despite the loss of health benefits incurred by the use of unsaturated oils, as is further described above. Therefore, it is further desirable that such modifications aimed towards improving the properties of edible fat-based materials would not alter other critical features, such as the degree of saturation of the oils contained therein and would not contain trans fats.

Thus, the need for compositions that are based on solid/semi-solid edible oils with high degree of unsaturation in food and medicinal/therapeutics production is well established in the art.

However, the prior art fails to describe an effective process for the preparation of such compositions, since the common process for solidifying unsaturated oils results in higher degree of saturation and formation of trans fats.

There is thus a widely recognized need for, and it would be highly advantageous to have, a thickened and/or thixotropic composition of an edible unsaturated oil, characterized by both semi-solid and liquid state, depending on temperature and/or the application of mechanical forces thereto, so as to address both the solid state requirement of the edible oils and also a high degree of unsaturation beneficiary in food and medicinal/therapeutic products.

### SUMMARY OF THE INVENTION

According to the present invention there are provided thickened oil compositions, in particular compositions that exhibit thixotropic properties, that are comprised of high proportions of edible oils, in particular unsaturated oils, and edible solidifying agents, in particular long chain fatty acids and/or long chain fatty alcohols, for use in the preparation of edible foods and medicinal/therapeutic products. Thus, according to the present invention there are provided edible food and medicinal/therapeutic products, processes for the preparation thereof and methods of consuming same, where such edible products include solid or semi-solid edible unsaturated oils, and therefore exhibit both quality and health benefits.

According to one aspect of the present invention there is provided a thickened oil composition comprising at least one edible oil and at least one edible solidifying agent, the edible solidifying agent being for converting the edible oil into the thickened oil composition.

According to another aspect of the present invention there is provided a process of producing a thickened oil composition, the process comprising (i) blending a major portion of at least one edible oil in combination with at least one edible solidifying agent at a temperature above the melting temperature of the at least one edible solidifying agent, so as to produce the thickened oil composition; and (ii) cooling the thickened oil composition to ambient temperature.

According to yet another aspect of the present invention there is provided an edible product comprising a base edible product or material and a thickened oil composition, the thickened oil composition including a combination of at least one edible oil and at least one edible solidifying agent, the edible solidifying agent being for converting the edible oil into the thickened oil composition.

According to still another aspect of the present invention there is provided a process of producing an edible product, the process comprising (i) preparing a thickened oil composition by blending a major portion of at least one edible oil with at least one edible solidifying agent; (ii) applying the thickened oil composition in substantially liquid form to a base edible product or material; and (iii) maintaining the thickened oil composition in substantially liquid form in contact with the base edible product or material for a time sufficient to permit at least a portion of the thickened oil composition to adhere to the base edible product or material.

According to an additional aspect of the present invention there is provided a food product comprising a base food product or material and a thickened oil composition, the thickened oil composition including at least one edible oil and at least one edible solidifying agent, the edible solidifying agent being for converting the edible oil into the thickened oil composition.

According to yet an additional aspect of the present invention there is provided a process of producing a food product, the process comprising (i) preparing a thickened oil by blending a major portion of at least one edible with at least one edible solidifying agent; (ii) applying the thickened oil composition in substantially liquid form to a base food product or material; and (iii) maintaining the thickened oil composition in substantially liquid form in contact with the base food product or material for a time sufficient to permit at least a portion of the thickened oil composition to adhere to the base food product or material.

According to still an additional aspect of the present invention there is provided a food product comprising a base food product or material and a modified fat substitute, wherein the modified fat substitute is a combination of a natural or synthetic fat-based food material and at least one edible solidifying agent, wherein the viscosity or melting temperature of the modified fat substitute is higher than that of the fat-based food material in the absence of the edible solidifying agent.

According to a further aspect of the present invention there is provided a process of preparing a modified fat substitute, the process comprising (i) selecting a natural or synthetic fat-based food material for which it is desirable to increase the viscosity or melting temperature; (ii) adding at least one edible solidifying agent to the natural or synthetic fat-based material; and (iii) blending, at the appropriate temperature, so as to produce a modified fat substitute having a higher viscosity or melting temperature than that of the fat-based food material in the absence of the edible solidifying agent.

According to yet a further aspect of the present invention there is provided a process of preparing a food product, the process comprising (i) selecting a natural or synthetic fat-based food material for which it is desirable to increase the viscosity or melting temperature; (ii) adding at least one edible solidifying agent to the natural or synthetic fat-based material; (iii) blending, at the appropriate temperature, so as to produce a modified fat substitute having a higher viscosity or melting temperature than that of the fat-based food material in the absence of the edible solidifying agent; and (iv) applying the fat-based food material onto a base food product or material.

According to still a further aspect of the present invention there is provided a liquid dairy product comprising a homogeneous mixture of substantially fat-free milk or milk powder dissolved in water and an edible thickened oil composition, the edible thickened oil composition including a combination of at least one edible oil and at least one edible solidifying agent, the edible solidifying agent being for converting the edible oil into the thickened oil composition, the liquid dairy product having a consistency and taste of fat milk.

According to another aspect of the present invention there is provided a solidified dairy product comprising a homogeneous mixture of substantially fat-free milk or milk powder dissolved in water and an edible thickened oil composition, the edible thickened oil composition including a combination of at least one edible oil and at least one edible solidifying agent, the edible solidifying agent being for converting the edible oil into the thickened oil composition, the solidified dairy product having a consistency and taste of solidified fat cheese. The solidified dairy product, according to the present invention has thixotropic properties and is therefore readily cut-through with a blade.

According to still another aspect of the present invention there is provided a semi-solid or solid dairy product comprising a homogeneous mixture of substantially fat-free milk or milk powder dissolved in water and an edible thickened oil composition, said edible thickened oil composition including a combination of at least one edible oil and at least one edible solidifying agent, said edible solidifying agent being for converting said edible oil into said thickened oil composition, the semi-solid or solid dairy product having a consistency of a conventional semi-solid or solid dairy product.

According to yet another aspect of the present invention there is provided a method of consuming therapeutically effective amounts of a therapeutic oil, the method comprising consuming an edible paste including a combination of at least one therapeutic edible oil and at least one edible solidifying agent, the edible solidifying agent being for converting the edible oil into the edible paste.

According to an additional aspect of the present invention there is provided a method of preparing an edible paste, comprising mixing at least one therapeutic edible oil and at least one edible solidifying agent, the edible solidifying agent being for converting the edible oil into the edible paste.

According to further features in preferred embodiments of the invention described below, the base edible product or material is selected from the group consisting of a food and an edible medicament.

According to still further features in the described preferred embodiments the thickened oil composition is externally applied onto the base edible product or material.

According to still further features in the described preferred embodiments the thickened oil composition is mixed with the base edible product or material.

According to still further features in the described preferred embodiments the thickened oil composition is a thixotropic composition, wherein the at least one edible solidifying agent converting the edible oil into the thixotropic composition, whereby the thixotropic composition exhibits a substantially semi-solid state upon rest at a temperature above the melting temperature of the at least one edible solidifying agent and a substantially liquid form upon being mechanically disturbed at the same temperature.

According to still further features in the described preferred embodiments the at least one edible solidifying agent includes an edible long chain fatty acid and/or an edible solidifying agent having a molecular weight of at least 200 Da and at least one hydroxyl group.

According to still further features in the described preferred embodiments the at least one edible oil is present at a concentration ranging from 50 to 99 percent by weight of the thickened oil composition.

According to still further features in the described preferred embodiments the edible long chain fatty acid has 18 to 50 carbon atoms in its hydrocarbon chain.

According to still further features in the described preferred embodiments the edible long chain fatty acid has at least one double bond in its hydrocarbon chain.

According to still further features in the described preferred embodiments the edible long chain fatty acid has at least one branched alkyl group side chain.

According to still further features in the described preferred embodiments the at least one edible oil comprises an unsaturated oil.

According to still further features in the described preferred embodiments the at least one edible oil comprises a major portion of an unsaturated oil.

According to still further features in the described preferred embodiments the unsaturated oil is present at a concentration range of from 70 to 99.8 percent by weight.

According to still further features in the described preferred embodiments the unsaturated oil comprises a major portion of a polyunsaturated oil.

According to still further features in the described preferred embodiments the at least one edible oil includes at least 6 percent by weight of an oil selected from the group consisting of omega-3 oil, omega-6 oil and a mixture thereof.

According to still further features in the described preferred embodiments the at least one edible oil is derived from a vegetable, a terrestrial animal, a marine origin or a combination thereof.

According to still further features in the described preferred embodiments the at least one edible oil is selected from the group consisting of palm seed oil, sal oil, mango seed oil, butter oil, cocoa oil, olive oil, soybean oil, canola oil, repaired oil, cottonseed oil, coconut oil, palm oil, sesame oil, sunflower oil, borage seed oil, syzigium aromaticum oil, hempseed oil, herring oil, cod-liver oil, salmon oil, corn oil, flaxseed oil, wheat germ oil, evening primrose oil, castor oil, linen oil, matthiola incana oil, garlic oil, shark oil, hypericum oil, sweet basil oil, mineral oil, silicone oil, highly saturated oils from animal sources and a mixture thereof.

According to still further features in the described preferred embodiments the edible product further comprising at least one edible excipient, selected from the group consisting of water, a surfactant, an emulsifier, a monoglyceride, a diglyceride, a triglyceride, an anti-oxidant, a stabilizer, a flavoring agent, a colorant agent and an odorant agent.

According to still further features in the described preferred embodiments the flavoring agent is selected from the group consisting of sugar, salt, pepper and cumin.

According to still further features in the described preferred embodiments the edible product further comprising at least one therapeutic agent in an amount up to 20 times the recommended daily dose per serving unit.

According to still further features in the described preferred embodiments the at least one therapeutic agent is selected from the group consisting of vitamin A, vitamin C, vitamin D, vitamin E, vitamin B1 (thiamin), vitamin B2 (riboflavin), niacine, nicotinamide, vitamin B6 IS (pyridoxine), vitamin B12, folic acid, biotin, panthothenic acid, inorganic salts or organometallic complexes of sodium, calcium, iron, iodine, magnesium, zinc, selenium, copper, manganese, chromium and vanadium, powder or extracts of aloe vera, anise, balm, baneberry, basil, thyme, bearberry, bloodroot, cedar, chamomile, chicory, cloves, echinacea, fennel, feverfew, garlic, ginseng, horse nettle, ivy, juniper, lavender, horse nettle, onion, parsley, pepper, rosemary, sage, saw palmetto berries, St. John's wrote, senna, thyme, valerian, yarrow and a mixture thereof.

According to still further features in the described preferred embodiments the edible solidifying agent having the molecular weight of at least 200 Da and at least one hydroxyl group comprises a fatty alcohol derivative having at least 15 carbon atoms in its hydrocarbon chain.

According to still further features in the described preferred embodiments the fatty alcohol derivative has up to about 50 carbon atoms in its hydrocarbon chain.

According to still further features in the described preferred embodiments the fatty alcohol derivative comprises an edible di-alcohol of formula A:

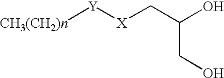

wherein, n=848;

X═CHR, O, or NH;

Y CUR; or C═O; and

R═H or alkyl.

According to still further features in the described preferred embodiments the fatty alcohol derivative comprises an edible di-alcohol of formula B:

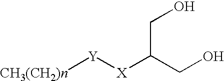

wherein, n=8-48;

X═CHR, O, or NH;

Y═CHR; or C═O; and

R═H or alkyl.

According to still further features in the described preferred embodiments the edible product further comprising a medicament.

According to still further features in the described preferred embodiments the at least one solidifying agent comprises a mixture of fatty alcohols derived from wax substances, such as beeswax, a majority of the fatty alcohols having at least 20 carbon atoms in their hydrocarbon chains.

The present invention successfully addresses the shortcomings of the presently known configurations by providing thickened or thixotropic oil compositions of edible unsaturated oils which are characterized by a substantially semi-solid state while at rest and a liquid form when thermally or mechanically disturbed, and thus provide solid or semi-solid edible oils with high degree of unsaturation that are used in accordance with preferred embodiments of the invention in the preparation of food and medicinal/therapeutic products.

### DESCRIPTION OF THE PREFERRED EMBODIMENTS

The present invention is of a thickened, preferably thixotropic, oil composition of an edible oil which can be used for the preparation of edible food and medicinal/therapeutic products. Specifically, the present invention can be used for the preparation of various edible products by using a vehicle, in the form of a thickened or a thixotropic oil composition of an edible unsaturated oil, for combining the ingredients required to produce acceptable edible product in which a homogeneous distribution of the ingredients will allow a simplification of commercial operations, where the edible products are easy to use and prepare and comprise high proportions of unsaturated oils to substitute for traditional saturated oils.

An important feature of the present invention relates to the flow properties of the compositions used to prepare edible food and medicinal/therapeutic products comprising, or coated with, unsaturated oil. Viscosity is the most commonly known term that describes flow properties. Often used as a single parameter to characterize materials, the viscosity that a product should have for a specific application is a complex topic as is 10 further addressed below.

Thus, according to the present invention, a thickened oil composition is a composition or compound which has been modified due to combination with a thickening, solidifying, or gelinating agent, thereby turning full-bodied, thicker, and either becoming more solid or semi-solid at a particular temperature, more viscous, having more retarded flow properties, becoming more adherent to particular surfaces, or a combination thereof.

A thixotropic composition, according to the present invention, is a type of thickened oil composition, in the sense that solidifying agents may convert a liquid or semi-solid compound or composition to a more solid state, for a given temperature state. However, a thixotropic compound or composition has the property of exhibiting both a substantially solid or semi-solid state upon rest and a substantially liquid form upon being mechanically disturbed. Thus, conversion of a liquid or semi-solid substance to become more thixotropic through addition of a solidifying, gelinating, or thickening agent means that the resultant mixture may exhibit two different types of physical states at the same temperature, depending upon its state of mechanical agitation.

The properties of enhanced thickness, enhanced viscosity, retarded flow rates, and/or thixotropic properties allow for application to an article, or surface of an article, of a thickened mixture as a liquid, which subsequently becomes substantially more solid than compared to an unthickened mixture, and therefore more adherent to the article or surface thereof.

Edible thickened oil compositions, when thickened or converted to thixotropic compositions due to combination with certain edible solidifying agents, are useful in the arts of food and medicine production. Low viscosity or thickness is recommended for easy spreading or coating of an article, but higher viscosity or thickness provides increased layer thickness for better coverage of area and conservation of mixture integrity. Thus, the combination of oil compositions with appropriate solidifying agents allows for the manipulation of Theological properties through use of the new combinations to achieve desirable results for the purpose of applying oil to articles, in particular food or medicinal/therapeutic articles, using the traditional processes of coating, spreading, spraying, layering, dipping, or combinations thereof.

The invention described herein discloses edible thickened oil-containing compositions, edible oil-containing products prepared from thickened or thixotropic oil compositions, and processes for the preparation thereof, that are particularly useful due to the inclusion therein of unsaturated oils in high proportions, while retaining or preserving the desirable properties attributed to traditional products containing high proportions of saturated oils.

Thus, according to one aspect of the present invention, there is provided a thickened oil composition of an edible oil.

The thickened oil composition of the present invention is comprised of an edible oil, one or more edible solidifying agent and, optionally, additional edible excipients, additives or mixtures thereof.

In particular, the composition of the present invention includes edible unsaturated oils, the weight concentration of which is variable, ranging between 20-40%; 30-50%; 40-60%; 50-70%; 60-80%; 70-90%; or 80.8%. More preferably, it is 50 to about 99.8% or 75 to about 99.8%. Preferably they are selected from a variety of oils from vegetable or marine sources. The addition of the thickening agent, at concentrations of about 1-30%, confers upon the parent oil properties by elevating its melting temperature and creating firmer consistencies.

Suitable unsaturated oils, according to the present invention, may be selected from the group consisting of olive oil, soybean oil, canola oil, rapeseed oil, cottonseed oil, coconut oil, palm oil, sesame oil, sunflower oil, borage seed oil, syzigium aromaticum oil, hempseed oil, herring oil, cod-liver oil, salmon oil, corn oil, flaxseed oil, wheat germ oil, evening primrose oil, castor oil, linen oil, matthiola incana oil, garlic oil, shark oil, hypericum oil, sweet basil oil, or mixtures thereof, at any proportion.

A particularly preferred class of oils includes poly-unsaturated oils, containing omega-3 and omega-6 fatty acids. Thus, in an especially preferred embodiment of the present invention the unsaturated oil contains at least 6% of an oil selected from omega-3 oil, omega-6 oil, or mixtures thereof.

Further according to the present invention, even oils that are solid or semi-solid at ambient conditions, due to high degree of saturation of the fatty acids thereof, can be improved by the addition of the solidifying agents of the present invention. Such oils may be from plant or animal sources. By way of example, such semi-solid or solid oils may be selected from the group consisting of palm oil, palm kernel oil, coconut oil, butter oil, milk fat and chicken fat. When applied at concentrations of about 1-10%, higher melting temperatures and/or firmer consistencies are attained.

Yet, another class of oils may include paraffin (mineral) oils and silicone oils.

Preferred solidifying agents, according to the present invention, include one or more edible long chain fatty acid and/or edible long chain fatty alcohols. Preferably, the edible solidifying agent of the present invention has a molecular weight of at least 200 Da and converts the composition of the oil and the solidifying agent into a thickened/thixotropic composition.

Edible long chain fatty acids, according to the present invention, are fatty acids having 18 or more carbon atoms in their hydrocarbon chain, such as stearic acid, arachidic acid (C20), behenic acid (C22), octacosanoic acid (C28), as well as fatty acids with longer hydrocarbon chains (up to C50), or mixtures thereof, which are allowed for human ingestion. The edible long chain fatty acids, according to the present invention, further include fatty acids having one or more hydroxyl group attached to the hydrocarbon chain thereof. The concentration of the fatty acid required to obtain a thickened oil-flavor system is inversely related to the length of its hydrocarbon chains. Stearic acid, for example, exerts a considerable thickening effect at about 10-20% concentration, whereas about 5-10% concentration is required to achieve the same thickening effect with behenic acid.

Edible long chain fatty alcohol, also referred to in the art as monomeric fatty alcohol and some times monomeric alcohols, according to the present invention, are fatty alcohols having 15 or more carbons in their hydrocarbon chain, such as cetyl alcohol and stearyl alcohol (or mixtures thereof), which are allowed for human ingestion without restriction (see, U.S. Food and Drug Administration, Center for Food Safety & Applied Nutrition; EAFUS: A Food Additive Database). Other examples of fatty alcohols are arachidyl alcohol (C20), behenyl alcohol (C22), 1-triacontanol (C30), as well as alcohols with longer hydrocarbon chains (up to C50). The concentration of the fatty alcohol, required to obtain a thixotropic oil-flavor system is inversely related to the length of its hydrocarbon chain.

Another class of solidifying agents according to the present invention comprises long chain fatty acids and/or long chain fatty alcohols having at least 18 carbon atoms and at least one double bond in their hydrocarbon chain.

A further class of solidifying agents according to the present invention comprises long chain fatty acids and/or long chain fatty alcohols, wherein the hydrocarbon chain is branched, containing at least one alkyl group side chain. By way of example, the alkyl group side chain can be a methyl, ethyl, n-propyl, i-propyl, n-butyl, t-butyl, i-butyl, n-pentyl, i-pentyl, t-pentyl, neo-pentyl, or linear or branched hexyl, heptyl, octyl, nonyl, decyl, lauryl, cetyl or stearyl group.

Yet, a further class of solidifying agents according to the present invention comprises long chain fatty acids and/or long chain fatty alcohols having at least one additional hydroxyl group linked to the hydrocarbon chain thereof.

Another class of solidifying agent according to the present invention comprises an edible di-alcohol, having formula A or B as follows:

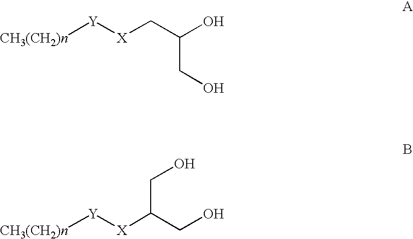

wherein, n is an integer ranging from 8 to 48; X is CHR, O, or NH; Y is CHR or C═O; and R is H or alkyl.

The solidifying agent of the present invention can be a component of a natural product such as beeswax, which can be used per se without the separation of the fatty alcohols and/or the fatty acids therefrom.

Thus, in preferred embodiments of the present invention the solidifying agent comprises wax substances, such as beeswax, or a mixture of fatty alcohols and/or fatty acids derived from wax substances, such as beeswax, a majority of the fatty alcohols and/or fatty acids having at least 20 carbon atoms in their hydrocarbon chains.

In especially preferred embodiments of the present invention, the oil-containing composition comprises, as the solidifying agent, a combination of at least one long chain fatty acid and at least one long chain fatty alcohol, which enables fine adjustment of the composition's Theological properties.

In a preferred embodiment of the present invention the thickened oil composition further comprises flavoring substances including water soluble, oil soluble and particulate flavors, commonly used for flavoring foods. In a similar fashion, colorants and odorants may be included in the thickened mixture, to produce an edible product with enhanced appeal.

In another preferred embodiment of the present invention the thickened oil composition farther comprises therapeutic agents. By way of example, therapeutic agents, suitable for inclusion in the edible thickened oil of the present invention may include vitamins A, C, D, E, B1(thiamin),B2 (riboflavin), niacine, nicotinamide, B6 (pyridoxine), B12, folic acid, biotin, panthothenic acid. They may also include minerals, such as sodium, calcium, iron, iodine, magnesium, zinc, selenium, copper, manganese, chromium and vanadium, any of them as inorganic salts or organometallic complexes. They may further comprise powdered medicinal/therapeutic herbs or herbal extract of aloe vera, anise, balm, baneberry, basil, thyme, bearberry, bloodroot, cedar, chamomile, chicory, cloves, echinacea, fennel, feverfew, garlic, ginseng, horse nettle, ivy, juniper, lavender, horse nettle, onion, parsley, peppermint, rosemary, sage, saw palmetto berries, St. John's wort, senna, thyme, valerian, yarrow, as well as other herbs known in the art of herbal medicine. Such therapeutic additives may be included in the composition at amounts of up to 20 times their respective recommended daily dose, per serving unit (when applicable).

Therapeutic agents according to the present invention may also consist of a prescription or non-prescription drug substance (hereinafter referred to as a medication or medicament). Incorporating a medication in the present thickened oil composition provides a novel delivery methods for such patients who cannot swallow pills and capsules, due to physiological limitations.

Thus, the present invention, in its especially preferred embodiments, concerns a thickened oil composition comprising an edible unsaturated oil, one or more edible solidifying agent as defined and an edible flavoring, colorant, odorant, nutritive, or medicinal/therapeutic component. The comp, being solid at room temperature, provides a suitable substitute for conventional saturated fats, which carry untoward health risks.

The present invention further provides a process for producing a thickened oil composition, comprising an edible oil, one or more edible solidifying agent as defined herein, and optionally additional excipients, additives, or mixtures thereof. The process comprises the steps of (i) blending a major portion of an edible oil in combination with one or more edible solidifying agent at a temperature above the melting temperature of the solidifying agent system so as to create a thickened oil composition of the oil and the solidifying agent; (ii) optionally, adding with agitation to the composition of step (i) food excipients, fatty alcohols, surfactants, emulsifiers, monoglycerides, diglycerides, triglycerides, anti-oxidants, stabilizers, flavors, colorants, odorants, herbal substances, therapeutic agents or medications; and, (iii) cooling the composition of step (i) or step (ii) to ambient temperature, to produce a thickened oil composition comprising an edible oil, one or more solidifying agent, and optionally additional excipients or additives.

Selected examples of thickened oil composition are the compositions obtained upon adding 4-10 grams of behenic acid or 4-10 grams of stearyl alcohol or combinations thereof to 100 grams of soybean oil and stirring the mixture to up to 180° C. until the acid or alcohol is completely dissolved in the oil; or adding 8-20 grams of behenyl alcohol to 100 grams of olive oil and stirring the mixture at 60° C.; or adding 0.2-1 gram of octacosanoic acid to 10 grams of canola oil at 60° C.

As is discussed hereinabove, a combination of at least one fatty acid and at least one fatty alcohol is particularly preferable in several systems, as it enables fine adjustment of the composition's rheological properties. One skilled in the art will readily appreciate the oil-solidifying agent combinations and blending conditions required for the formation of the thickened oil composition comprising unsaturated oils which is a feature of the food products and process for preparation thereof disclosed herein.

In a further embodiment, the thickened composition of the present invention, prepared from an edible oil and one or more edible solidifying agent, is a thixotropic composition. The thixotropic composition exhibits both an at least semi-solid state upon rest and a liquid form upon being mechanically disturbed, at a temperature above the melting temperature of the edible solidifying agent. This thixotropy property of the composition is advantageous in several uses. The consistency of the thixotropic composition can be further modified by adding surfactants, emulsifiers, monoglycerides, diglycerides, triglycerides, and other edible excipients, normally used in the art of food production. Such modifications can be used to adjust the consistency of the composition by reducing the degree of thixotropy thereof, while conserving the desirable viscosity.

According to the present invention, the thickened oil composition, comprising an edible oil, one or more edible solidifying agent and, optionally, excipients and/or additives, described hereinabove, is used for the preparation of edible products. The edible products, according to the present invention, can be coated and/or flavored food products, modified fat substitutes and edible pastes for delivery of therapeutic amounts of oils to the human body. Thus, further according to the present invention, there are provided: (i) a coated and/or flavored food product that comprises a thickened oil composition; (ii) an edible spread that comprises a thickened oil composition as a primary component; (iii) a dairy food product that comprises high proportions of unsaturated oils to substitute for animal-originated fats; (iv) a food product that comprises a thickened composition of an edible fat-based material; (v) an edible therapeutic or medicinal product that comprises high proportions of therapeutic oils having a semi-solid or solid consistency; and (vi) an edible therapeutic or medicinal product that comprises a thickened oil composition and a therapeutic/medicinal agent; and processes of the preparation thereof.

Further according to the present invention, there are provided: (i) a food product that comprises a thickened oil composition and/or thixotropic oil composition of the present invention, and easy to use prepare; (ii) a food product which comprises high proportions of unsaturated oils to substitute for traditional saturated oils; (iii) a process for producing a food product that comprises thickened oil compositions and thixotropic oil compositions; (iv) a process for producing a food product which reduces oil spills in comparison with liquids oils currently in use for food production; (v) acceptable food products prepared from a process which enables the use of thickened oil compositions comprising intact unsaturated oils, which carry the healthy properties of such oils; (vi) an oil-flavor system for food production, comprised from thickened oil compositions, wherein salt and other flavoring particles are substantially distributed throughout the thickened oil composition, which is amenable to facile application to food materials or products; (vii) a process for producing a food product comprising an oil coating, wherein such a food product can benefit from being coated by an oil which enhances food preservation or protection and which comprises the health benefits of unsaturated oils; (viii) unsaturated oil compositions which are improved with respect to conventional semi-solid and solid fats, allowing food process to be performed at lower temperatures; and (ix) compositions and food products thereof which are useful, when supplemented with additives, for the administration of dietary supplements, vitamins, medications, and other therapeutic agents.

The process for food preparation disclosed herein utilizes the thickened oil composition of unsaturated oils of the present invention. Such oil compositions, when agitated, has low viscosity, and thus allowing facile application thereof onto food by pouring, sprinkling or spraying. Soon after application it becomes very viscous, which eliminates dripping and sedimentation of the oil and allows maintaining of flavors and therapeutic additives attached to the food. Thus, the thickened oil composition of the present invention is better than conventional solid or semi-solid fat compositions, which solidify gradually, thus requiring high temperature in order to facilitate application onto a food product and homogeneous distribution on the food and chilling in order to conserve consistency. Coating a food product with thickened oils can be achieved, for example, by spraying the oil composition onto a food product or by dipping a food product in the oil composition, either at ambient temperatures of at elevated temperature, up to 240° C.

Moreover, according to the method of the present invention, the chemical structure of the unsaturated oils is not changed, and therefore the health benefits, which are attributed to such oils are conserved, thus providing an alternative for current methods, which involve hydrogenation, yielding saturated fats, which carry untoward health risks.

One aspect of the present invention is to provide an effective, homogeneous oil-flavor coating of frozen food. Oils, such as olive oil and other different flavoring agents are often added to flavor such foods. Yet, it is technically difficult to apply oils and flavors on foods and to ensure that the oil does not drip from the food to the bottom of the package, prior to freezing. The composition and method of the present invention provide a technical solution for the above problem. When the thickened oil system is applied onto foods, by sprinkling, dipping or brushing, uniform distribution of the oil and flavoring is attained and no spillage occurs. Thus, according to another aspect of the present invention, there is provided a method for producing a food product, wherein the food product comprises the thickened oil composition of the present invention, the process comprises the steps of: (i) blending a major portion of an edible oil in combination with one or more edible solidifying agent so as to produce the thickened oil composition; (ii) optionally, adding with agitation to the composition of step (i) food excipients, surfactants, emulsifiers, monoglycerides, diglycerides, triglycerides, antioxidants, stabilizers, flavors, colorants, odorants, herbal substances, therapeutic agents, medications, or mixtures thereof; (iii) applying the composition of step (i) or step (ii) in liquid form to a base food product or material; and (iv) maintaining the composition of step (iii) in liquid form in contact with the base food product or material for a time sufficient to permit at least a portion of the blend to adhere to or be introduced into the base food product or material, so as to produce a food product comprising the thickened oil composition.

In a further embodiment, wherein the thickened oil composition is a thixotropic composition, the food product comprises the thixotropic composition of the present invention and the process comprising the steps of: (i) blending a major portion of an edible oil in combination with one or more edible solidifying agent, so as to produce the thixotropic composition, exhibiting a substantially semi-solid state upon rest and a liquid form upon being mechanically disturbed, at a temperature above the melting temperature of the solidifying agent; (ii) adding with agitation to the blend of step (i) food excipients, surfactants, emulsifiers, monoglycerides, diglycerides or triglycerides, antioxidants, stabilizers, flavors, colorants, odorants, herbal substances, therapeutic agents, medications, or mixtures thereof; (iii) applying the composition of step (ii) in liquid form to a base food product or material; and (iv) maintaining the composition of step (iii) in liquid form in contact with the base food product or material for a time sufficient to permit at least a portion of the composition to adhere to or be introduced into the base food product or material, so as to produce a food product comprising the thixotropic composition.

Thus, the present invention further concerns a method for applying flavoring to a food by preparing an oil-flavor mixture, having thickened properties and applying it on a food. The method is simple and convenient, produces an even flavor distribution, and in contrast to prior known methods for applying flavorings, does not require applying the flavoring while cooking the food in a liquid fat.

As described hereinabove, a thickened oil system is prepared by blending an edible unsaturated oil, an edible solidifying agent as defined herein and edible flavoring, at temperature above the melting temperature of the solidifying agent. The oil-flavor system is applied to the base food, for example, by shaking thereon, sprinkling or spraying. The oil-flavor system is maintained in contact with the base food for a time sufficient to permit at least a portion of the oil-flavor system to adhere to the base food.

In a preferred embodiment, the method is useful for producing flavored pastry, vegetables, potatoes, pasta, rice, or meats having an evenly distributed flavor, using the thickened oil system of the present invention. The thickened oil system preferably contains 75 to 99.8% by weight of one or more non-hydrogenated, unsaturated vegetable oils, such as of olive oil, soybean oil, canola oil, rapeseed oil, cottonseed oil, coconut oil, palm oil, sesame oil, sunflower oil, borage seed oil, syzigium aromaticum oil, hempseed oil, herring oil, cod-liver oil, salmon oil, corn oil, flaxseed oil, wheat germ oil, evening primrose oil, or mixtures thereof.

In another preferred embodiment, the method is useful for producing flavored popcorn having an evenly distributed flavor, using the thickened oil-flavor system of the present invention. Thus, in accordance with the present invention there is provided a composition for popping popcorn comprising a thixotropy oil-flavor system as defined herein, having salt and other flavoring particles homogeneously throughout. Preferably, the composition also includes coloring and flavoring. The present invention provides a system which is improved from the following viewpoints: (a) to provide a vehicle for combining the ingredients required to produce acceptable popcorn in which a homogeneous distribution of the ingredients will allow a simplification of corn popping in commercial operations; (b) to provide a product that is easy to use and to eliminate the possibility of oil spills in comparison with liquids oils currently in use for the popping of corn; and (c) to provide an oil composition for popping popcorn, containing intact unsaturated oils, which carry the healthy properties of such oils.

Yet, another object of the present invention is to provide an effective even oil-flavor coating of food products.

Thus, according to still another aspect of the present invention there are provided food products having a thickened unsaturated oil composition applied thereto, the composition comprising a major portion of an edible unsaturated oil in combination with one or more edible solidifying agent, as defined hereinabove.

As stated, in especially preferred embodiments of the present invention, the edible solidifying agent comprises at least one edible long chain fatty acid derivative and/or edible long chain fatty alcohol derivative.

In another especially preferred embodiment, the edible solidifying agent comprises at least one fatty acid derivative having at least 18 carbon atoms in its hydrocarbon chain.

In another especially preferred embodiments of the present invention, the edible solidifying agent comprises at least one fatty alcohol derivative having at least 15 carbon atoms in its hydrocarbon chain.

Preferably, the food product is selected from the group consisting of popcorn, pastry, vegetables, fruits, potatoes, pasta, rice, meats, meat substitutes, sausage and fish.

In a further preferred embodiment, the method is useful for delivering therapeutic substances, otherwise difficult to deliver for oral consumption, by incorporating them in a thickened oil-flavor system, as defined herein. The thickened oil-flavor system, containing such therapeutic substances, is further applied onto foodstuff, selected from popcorn, pastry, vegetables, fruits, potatoes, pasta, rice, fish, meats, meat substitutes and sausages.

According to yet another aspect of the present invention, there are provided: (i) food products comprising base food materials or products and modified fat substitutes; (ii) methods for the preparation thereof; and (iii) food products that are improved through the use of such modified fat substitutes in combination with traditional food materials or products.

It is an object of this aspect of the present invention that the modified fat substitutes employed therein are suitable for use in the preparation of a wide variety of food products.

A food substance is any substance considered suitable for human or animal consumption. The food products disclosed herein comprise food substances wherein one or more of the modified fat substitutes of the invention has been introduced as an addition to, or as a substitute for, one or more elements of the food material. For example a food substance includes any vegetable, fruit, meat, fish, egg, bean, seed, or dairy edibles which may be consumed raw, after cooking, or following any other acceptable form of processing, and which range from apple pie and ice cream to cheese burgers and fries. Other examples of food material include fresh, processed, or pre-cooked starch, carbohydrate, protein, fats, potatoes, wheat, corn, barley, oats, rye, millet, vegetables, fruit, tubers, legumes, leafy plants, marine animals, marine plants, nuts, beans, seeds, rice, milk, cheese, cream, yogurt, almonds, poultry, herbs, spices, and mixtures thereof.

The incorporation through the addition and blending of solidifying agents such as certain monomeric long chain fatty alcohols (herein “fatty alcohols”) together with edible fat-based materials such as margarines, shortenings, lards, and cooking oils leads to the formation of a new composition with physicochemical properties that are improved for consumption and manifest other advantageous properties, particularly in the field of food processing. As described hereinabove, compositions of edible fat-based materials, comprising saturated, unsaturated, or polyunsaturated oils in combination with a solidifying agent such as fatty alcohol, exhibit in general higher viscosities and melting temperatures than that of the parent fat-based material alone. This new property facilitates the food production process and provides for the production of food products with enhanced textural and stability properties.

Examples of natural or synthetic fat-based food materials are margarine, butter, cooking oil, edible oil of marine, vegetable or animal origin and lard.

Thus, the addition of solidifying agents such as fatty alcohol compounds of a hydrocarbon content ranging from 15 to 50 carbon atoms, as either heterogeneous or homogenous mixtures of these compounds, to edible fat-based materials, as described in the forthcoming tables and examples, results in compositions of greater consistency and which melt at higher temperatures. The blending of these components is readily apparent to one skilled in food production and the appropriate conditions, including temperature ranges for the blending phase.

Preferably, the solidifying agent with respect to this aspect of the invention is an edible long chain fatty alcohol derivative. Thus, fatty alcohols in the range of C16-C22, such as stearyl alcohol, are adequate at concentrations of 0.5-16% (w/w in the oil/alcohol mixture), while lower concentrations ranging from 0.1-6% are suitable when employing longer chain fatty alcohols, e.g., C-23-C50. Such fatty alcohols may be produced synthetically or derived from natural sources (e.g., beeswax and sugar cane) and other natural edible substances which contain high percentages of fatty alcohols (e.g., natural waxes).

In many cases, it is advantageous to cool the mixtures prior to further applying the compounds to further applications of the food production process.

The compositions of fatty alcohols with edible fat-based materials thus obtained may be suitably packaged and stored for future use or applied without additional delay in a continuous food production process. When stored and packaged, the compounds manifest the benefits of enhanced solidity including the possibility of packaging as solids, rather than liquids, a feature which facilitates, among other advantages, a wider choice of packaging, measuring, storage, and transporting techniques. Thus, the modified fat substitute compositions described herein are useful as food products which serve as substitutes for the same fat-based materials which, as food products, are typically stored, transported and distributed for use in food processing in both domestic and commercial venues. Thus, the present invention provides for modified fat substitutes which may directly replace, when desirable, the conventional fat-based materials, such as margarine, lard, shortening, and cooking oils used as starting materials in a wide-variety of food processing applications, including the production of creams, spreads, batters, pastes, sauces and in the processes of cooking such as baking, frying, broiling, boiling, and irradiating food materials, whether as raw or pre-processed ingredients.

In addition to the enhanced consistency and melting temperatures offered by the use of the modified fat substitutes described herein, these compositions provide for additional advantages. In particular, the oil components of the parent fat-based material are not substantially altered with respect to their chemical structures, thus maintaining the health benefits bestowed by these ingredients. This feature is especially significant in the case of unsaturated oil components, which are considered to be preferred ingredients of food with respect to the cardiovascular risk factors inherent to oil consumption. By not containing trans fats, such food products do not possess the safety hazards, which are currently being attributed to solidified oils which are produced by hydrogenation processes. In addition, the conversion of liquid or semi-solid edible fat-based materials to more solid states, may expectedly lead to enhanced commercial value with regard to facility of storage, handling, and transport, when employed as fat substitute in a food product or the preparation thereof.

A wide range of fat-based materials may be chosen for implementation of the present invention, including fat-based materials comprising oils consisting of saturated, mono-unsaturated, or poly-unsaturated oils, derived from plant, animal, or marine sources. Thus, according to the present invention, the production of modified fat substitutes is accomplished from fat-based materials comprising oils such as, but not exclusively, palm oil, palm seed oil, sal oil, mango seed oil, cocoa oil, olive oil, corn oil, soybean oil, canola oil, cottonseed oil, coconut oil, sesame oil, sunflower oil, safflower oil, borage seed oil, syzigium aromaticum oil, hempseed oil, herring oil, cod-liver oil, salmon oil, flaxseed oil, wheat germ oil, evening primrose oil, highly saturated oils from animal sources, or mixtures thereof. When desirable, modified or synthetic oils are similarly applicable to implementation of the invention.

In one embodiment, fat-based materials for preparation of the modified fat substitute of the invention, can be further processed by fatty alcohols of the present invention to obtain higher melting temperatures and better properties with respect to food production.

Blending oils and fat-based food materials comprised thereof with fatty alcohol derivatives results in modified fat-based materials which bear improved physicochemical characteristics for food production. Fatty alcohol compounds which may be chosen for this process are, in general, composed of from 15 to 50 carbon atoms and having at least one hydroxyl group in their structure. The alcohols may be derived from both natural and synthetic sources, the sole limitation being edible materials and having the ability to produce modified fat substitutes with improved consistency or melting temperatures when appropriately blended with the parent fat-materials.

Thus, preferred fatty alcohol derivatives used as solidifying agents of the modified fat substitute of the invention are as described hereinabove, with respect to the thickened oil composition of the invention. The fatty alcohol composition of the modified fat substitute may range from 0.1 to 15 %.

In a preferred embodiment of this aspect of the present invention the modified fat substitute composition or the food product produced therefrom further comprise flavoring substances including water soluble, oil soluble and particulate flavors, commonly used for flavoring foods. In a similar fashion, colorants and odorants may be included in the fat substitute or food products to produce a food product with enhanced appeal.

In another preferred embodiment of this aspect of the present invention, the fat substitute or food product derived therefrom further comprise therapeutic agents which are defined hereinabove. The therapeutic additives may be included at amounts of up to 20 times their respective recommended daily dose, per serving unit, as desired. The therapeutic agents according to this aspect of the invention may also consist of a medication, as is defined hereinabove.

Thus, the present invention, in its especially preferred embodiments, concerns modified fat substitute compositions and the food products produced therefrom, comprising an edible fat-based material and an edible fatty alcohol as defined, in combination with an edible flavoring, colorant, odorant, nutritive, or medicinal/therapeutic component. The food products, being more solid at room temperatures, provides sustain the advantages of enhanced stability and texture, while offering the options of employing less oil, or unsaturated oils, thereby obviating some of the untoward dietary risks associated with many fat-based materials.

In another especially preferred embodiment of the present invention, the modified fat substitute composition further comprises an emulsifier system or a highly saturated triglyceride, both of which may be incorporated to improve the consistency of the composition and to enable facile incorporation of said composition with other food constituents.

Further provided by the invention is a method for producing a food product comprising a modified fat substitute, the method comprising the steps of: (i) selecting a natural or synthetic fat-based food material, for which it is desirable to increase the viscosity or melting temperature of said material; (ii) creating a modified fat-based food material wherein a monomeric fatty alcohol is added to the natural or synthetic fat-based material selected in step (i); (iii) optionally, adding desired flavor, colorant, odorant or therapeutic agent, emulsifier system, admixture of highly saturated triglycerides, or a combination thereof; (iv) blending, at the appropriate temperature, the modified fat-based material of step (ii) or step (iii), so as to produce a fat substitute having a higher viscosity or melting temperature than that of the material in the absence of the fatty alcohol; (v) alternatively, allowing the product to cool; (vi) still alternatively, applying the conventional steps of food preparation to the product of the preceding steps, either alone or by combination with additional food substances or materials to produce a food product comprising a modified fat substitute.

In a preferred embodiment, the method for preparation of a food product comprising a modified fat substitute is useful for producing popcorn, pastry, vegetables, fruits, potatoes, pasta, pizza, rice, meats, fish, meat substitutes and sausages having a substantial proportion of said modified fat substitute as an addition or replacement for the parent or traditional fat-based material of the desired food in preparation. Preferably, according to the present invention, the food product contains from 0.5 to 99.8% by weight of modified fat substitute, wherein one or more of the vegetable, animal or marine oils described herein are present in said substitute in a range of 50 to 99.8% and the fatty alcohol content of said substitute ranges from 0.1 to 15%.

In preferred embodiments, the modified fat substitute of the invention comprises at least 50% of an oil selected from the group consisting of palm, palm seed, sal, mango seed, butter, cocoa, olive, corn, soybean, canola, cottonseed, coconut, sesame, sunflower, borage seed, syzigium aromaticum, hempseed, herring, cod-liver, salmon, flaxseed, wheat germ, evening primrose oils, highly saturated oils from animal sources, and mixtures thereof.

In especially preferred embodiments, the modified fat substitute obtained comprises, alternatively or in addition, at least 50% of an oil selected from the group consisting of omega-3, omega-6 fatty acids, and mixtures thereof.

The modified fat substitute and the process for food production provided herein may be applied to a wide variety of food products and processing techniques, ranging from heated and fried articles to cooled gels and ice creams. Thus, the present invention provides for margarine, butter, shortening, cream, sauce, dressing, spreads, and combinations thereof; food products selected from group consisting of flour, wheat, bran, barley, corn, rice, oats, milk, eggs, fats, oils, vegetables, meats, fish, fruit, nuts, and combinations thereof; food products selected from the group of raw or uncooked fat-based food materials, and combinations thereof; food products selected from the group consisting of bread, doughnuts, sauces, dressings, coatings, spreads, salad dressings, margarines, butters, peanut butter, almond butter, cooking oils, cheeses, pastes, cakes, pastries, muffins, bagel, biscuits, pizza, sesame spread, chick pea spread, mustard, confections, comestibles, re, ice creams, yogurts, frozen yogurt, filling creams, waffles, and chocolate substitute; food products selected from the group consisting of fresh, processed, or pre-cooked starch, carbohydrate, protein, fats, potatoes, wheat, corn, barley, oats, rye, millet, vegetables, fruits, tubers, nuts, beans, seeds, rice, fish, eggs, milk, cheese, meats, meat substitutes, sausages, poultry and mixtures thereof; and food products selected from the group consisting of fresh, processed, or pre-cooked popcorn, pastry, breads, baked goods, pasta, batter, crackers, potato chips, tortilla, snack foods, confections, comestibles and mixtures thereof. Similarly, the process of the invention, which discloses the use of modified fat substitutes in food preparation, entails the acts employing the modified fat substitute in the art of food processing, including the acts of mixing, blending, pouring, coating, pouring, shaping, inserting, dipping, layering, heating, cooking, boiling, frying, baking, broiling, irradiating, and combinations thereof. Furthermore, the invention provides for food products manufactured by the foregoing process.

An example of a simple and most straightforward use of the modified fat-substitute of the present invention is in the production of shortenings. Shortenings are semi-solid and solid fats, used as the fat component of many food products. Traditionally, a shortening is prepared from partially or fully hydrogenated vegetable oils. The hydrogenation process involved in the production of traditional shortenings results in the formation of saturated fats and trans-fatty acids, the consumption of which is linked to elevated LDL cholesterol blood levels and higher risk of cardiovascular damage, as is further discussed hereinabove. By contrast, the modified fat-substitute of the present invention is a semi-solid and/or solid composition that includes limited amounts of saturated fats and no trans-fats, thus providing a healthier alternative.

Thus, according to the present invention there is provided a liquid dairy product comprising a homogeneous mixture of substantially fat-free milk or milk powder dissolved in water (skimmed milk or milked powder) and an edible thickened oil composition, the edible thickened oil composition including a combination of at least one edible oil and at least one edible solidifying agent, the edible solidifying agent being for converting the edible oil into the thickened oil composition, the liquid dairy product having a consistency and taste of fat milk.

Further according to the present invention there is provided a solidified dairy product comprising a homogeneous mixture of substantially fat-free milk or milk powder dissolved in water and the edible thickened oil composition of the present invention. The solidified dairy product of the present invention has a consistency and taste of solidified fat cheese, which is readily cut-through with a blade due to thixotropic properties thereof.

Further according to the present invention there is provided a semi-solid or solid dairy product comprising a homogeneous mixture of substantially fat-free milk or milk powder dissolved in water and the edible thickened oil composition. The semi-solid or solid dairy product of the invention has a consistency of a conventional semi-solid or solid dairy product.

By way of example, the dairy products of the present invention can be fat milk, yogurt, butter, buttermilk, milk powder, mozzarella cheese and quark cheese.

The liquid, solid and semi-solid dairy products of the present invention demonstrates the advantageous use of the modified fat-substitutes of the present invention by providing fat-based dairy products where the conventional solid or semi-solid fats are substituted by a major portion of unsaturated oils.

According to further aspects of the present invention, there are further provided: (i) an edible paste comprising a therapeutic oil and at least one solidifying agent; (ii) a method for preparing the edible paste; and (iii) a use of the edible paste for delivery of therapeutic amounts of a therapeutic oil.

The use of unsaturated oils having a therapeutic value in the preparation of medicinal/therapeutic products is limited due to the form thereof. Ingesting such oils by spoon is limited due to their unacceptable taste and/or odor, while the commonly used soft gel capsules, which hinder the taste oily sensation of the oils, do not provide a satisfactory alternative, since they contain limited amounts of oil, in the range of 0.2 to 0.5 gram, which is, in many cases, far less than the daily-recommended therapeutic amount as is shown in Table 1 herein below. Swallowing many such capsules per day on a continuous basis is not practical. Larger capsules (up to 1.5 ml) are in existence, but they are hard to swallow.

In order to obviate this problem, it has now been discovered that it is possible to provide therapeutic oils in the form of edible pastes, preferably with the addition of flavoring additives, and thereby to overcome this long standing resistance to the proper and extensive use of therapeutic oils.

Thus, the present invention provides for an edible paste for delivery of therapeutic amounts of therapeutic oils, comprising a combination of at least one therapeutic edible oil and at least one edible solidifying agent, wherein the edible solidifying agent being for converting the edible oil into the edible paste.

According to a preferred embodiment of this aspect of the present invention, the edible paste is a thixotropic composition as is defined hereinabove.

According to another preferred embodiment of this aspect of the present invention, the therapeutic oil is an unsaturated oil. As used herein, the unsaturated oils are liquid oils derived from vegetable or marine origin, that include at least 70% unsaturated fatty acids, such as, but not limited to, evening primrose oil, castor oil, syzigium aromaticum oil, linen oil, matthiola incana oil, flaxseed oil, hempseed oil, garlic oil, herring oil, salmon oil, cod liver oil, shark oil, sunflower oil, olive oil, hypericum oil, borage seed oil and sweet basil oil.

Especially suitable unsaturated oils are those including at least 12% fatty acids that have two or more double bonds. Thus, according to a preferred embodiment of this aspect of the invention, the therapeutic oil includes at least 6 percent by weight of an oil selected from the group consisting of omega-3 oil, omega-6 oil and a mixture thereof.

Other therapeutic oils that can be used while implementing this aspect of the present invention are mineral oils, silicon oils and a mixture thereof.

The weight percentage of the therapeutic oil included in the edible paste of the present invention can range from 50% to 99.8%, preferably from 80% to 99.8%, more preferably from 95% to 99.8%, most preferably more than 99%.

The edible solidifying agent, according to this aspect of the present invention, includes one or more edible long chain fatty acid, as is defined hereinabove, and/or edible solidifying agent having a molecular weight of at least 200 Da and at least one hydroxyl group.

According to a preferred embodiment, the edible solidifying agent is a fatty alcohol, as is defined hereinabove. Suitable fatty alcohols are, for example, cetyl alcohol and stearyl alcohol, behenyl alcohol, 1-triacontanol and fatty alcohols with longer hydrocarbon chain (up to C50).

According to another preferred embodiment of this aspect of the present invention, the weight percentage of the fatty alcohol in the edible paste is less than about 2%.

The edible paste of the present invention can further comprise solid components with specific gravity of between 0.6 and 1.4 that are evenly distributed in the edible paste. Especially preferred solid components include spices and powdered herbal materials.

According to a preferred embodiment of this aspect of the present invention, the edible paste can further comprise one or more edible excipient, such as water, surfactant, emulsifier, monoglyceride, diglyceride, triglyceride, and flavoring, colorant or odorant agent.

In especially preferred embodiments of this aspect of the present invention, the edible paste further comprises flavoring additives.

The edible paste of the present invention can further comprise a therapeutic agent and/or a medicament as is defined hereinabove.

Thus, according to the present invention, there is provided as a preferred embodiment, an edible paste for use as a lipid lowering remedy, comprising: (a) 80 to 99.8 percent by weight of an omega-3 oil; and (b) an edible solidifying agent that comprises a fatty alcohol derivative having at least 15 carbon atoms in its hydrocarbon chain.

According to another preferred embodiment, there is provided an edible paste for use as a diabetes remedy, comprising: (a) 80 to 99.8 percent by weight of an omega-3 oil; (b) an edible solidifying agent that comprises a fatty alcohol derivative having at least 15 carbon atoms in its hydrocarbon chain; and (c) up to 1 percent by weight of at least one trace mineral, selected from vanadium, chromium, tungsten, zinc or a mixture thereof.

According to still another preferred embodiment, there is provided an edible paste for use as a constipation remedy, comprising: (a) 80 to 99.8 percent by weight of an omega-3 oil; and (b) an edible solidifying agent that comprises a fatty alcohol derivative having at least 15 carbon atoms in its hydrocarbon chain.

According to still another preferred embodiment, there is provided an edible paste for use as a hyperactivity remedy, comprising (a) 80 to 99.8 percent by weight of an omega-3 oil; and (b) an edible solidifying agent that comprises a fatty alcohol derivative having at least 15 carbon atoms in its hydrocarbon chain.

The present invention further provides for a method of preparing the edible paste of the present invention, the method comprising mixing at lone therapeutic edible oil and at least one edible solidifying agent, the edible solidifying agent being for converting the edible oil into the edible paste.

The present invention further provides for a method of consuming therapeutically effective amounts of a therapeutic oil, the method comprising consuming the edible paste of the present invention, including a combination of at least one therapeutic edible oil and at least one edible solidifying agent, the edible solidifying agent being for converting the edible oil into the edible paste.

By way of example, the following Table 1 provides a list of therapeutic oils, their function and the recommended amount of grams per day necessary to achieve the therapeutic function attributed thereto.

TABLE 1

| | | Amount |
|---|---|---|
| Therapeutic oils | Target disorder / Function | (gr/day) |
| Evening primerose | Hyperaction | 1-4 |
| | Coagulation disorders | |
| | Cardiovascular disorders | |
| | Antiinflammatory | |
| Flaxseed | Hypercholesteremia, hypertriglyceride | 1-4 |
| Castor oil (Ricinus Communis) | Laxative | 1 |
| Syzigium Aromaticum | Digestive disturbances | 1 |
| Garlic | Antibacterial | 1 |
| | Anticoagulant | |
| | Anti-hypertensive | |
| | Anti-cholesterol | |
| Herring, Salmon | Anti-lipidemic | 4 |
| | Anti-hypertensive | |
| | Cancer protection | |
| Cod Liver | Anti-lipidemic | 4-6 |
| | Anti-cholesterol | |
| | Anti-hypertensive | |
| | Anti-inflammatory | |
| | Cancer protection | |
| Shark | Anti-cancer | 2 |
| Sunflower | Protection against drug-induced | 4-20 |
| | nephrotoxicity | |
| Olive | Anti-lipidemic | 4 |
| | Anti-cholesterol | |
| | Anti-hypertensive | |
| | Cancer protection | |
| Hypericum | Anti-depressive | 1 |
| Borage Seed | Immunomodulation | 2 |
| St. John's wort | Depression | 2 |
| Sweet Basil (Basilicum) | Anti-inflammatory | 1 |

Table 1 demonstrates the advantages of the use of the edible paste of the present invention for delivery of therapeutic amounts of therapeutic oils.

Additional objects, advantages, and novel features of the present invention will become apparent to one ordinarily skilled in the art upon examination of the following examples, which are not intended to be limiting. Additionally, each of the various embodiments and aspects of the present invention as delineated hereinabove and as claimed in the claims section below finds experimental support in the following examples.

### EXAMPLES

Reference is now made to the following examples, which together with the above descriptions, illustrate the invention in a non-limiting fashion.

### Example 1

### Production of Beeswax-Derived Fatty Alcohols

Twenty grams of beeswax were added to 50 grams of ethanol at 60° C with stirring. A large amount of foam was generated during this process. When the beeswax was completely dissolved in the ethanol, the mixture was allowed to cool off. Upon cooling, a white solid layer was formed at the bottom of the flask. The alcohol layer was decanted and the remaining solid was washed with water, to yield 9 grams of product. Gas chromatography-mass spectroscopy (GCMS) analysis revealed that the product comprised a mixture of long chain alcohols, having a general formula of CₙH₂ₙ₊₁-OH, in the following composition (Table 2):

TABLE 2

| n | Relative intensity |
|---|---|
| 23 | 3.0 |
| 25 | 4.1 |
| 27 | 3.0 |
| 29 | 0.8 |
| 31 | 2.0 |
| 33 | 4.9 |

### Example 2

### Thickened Edible Olive Oil-Flavor System

Eight grams of behenic acid were added to 90 grams of olive oil and the mixture was stirred at 60° C. until the acid was completely dissolved in 5 the oil. 0.3 gram of garlic extract, 0.5 gram salt and 0.2 gram ground majoran were added to the hot mixture and the mixture was cooled to 30° C. with stirring, then poured into containers and cooled to room temperature. A gelatin-like system was obtained, with taste and smell similar to the original olive oil. Upon shaking at 30° C., the gel became liquid and after application on food surface it became solid again.

### Example 3

### Thixotropic Edible Olive Oil-Flavor System

The method of Example 2 was applied, using 8 grams of stearyl is alcohol instead of behenic acid. Results were similar.

### Example 4

### Thickened Edible Fish Oil-Flavor System

Four grams of arachidic acid were added to 96 grams of cod liver oil and the mixture was stirred at 60° C. until the acid was completely dissolved in the oil. 0.5 gram of lemon concentrate, 0.4 gram salt, 0.1 gram black pepper and 0.2 gram ground garlic were added to the hot mixture and the mixture was cooled to 30° C. with stirring, then poured into containers and cooled to room temperature. A gelatin-like system was obtained. Upon shaking at 30° C., the gel became liquid and after application onto food surface it became solid again. GCMS analysis revealed that the lipid composition of the spread was identical to the original cod liver oil. The taste and smell of the resulting spread were distinctly different from the parent oil and were found acceptable by a panel of tasters.

### Example 5

### Thixotropic Edible Fish Oil-Flavor System

The method of Example 4 was applied, using 2 grams of beeswax-derived fatty alcohols instead of arachidonic acid. Results were similar.

### Example 6

### Thickened Edible Canola Oil-Flavor System with Therapeutic Agents

0.4 gram of behenic acid was added to 9.6 grams of canola oil and the mixture was stirred at 80° C. until the acid was completely dissolved in the oil. Vitamins, minerals and herbs were added to the hot mixture in order to achieve the following composition (Table 3):

TABLE 3

| | | | | | |
|---|---|---|---|---|---|
| Vitamin A | 20,000 | IU | Sodium chloride | 400 | mg |
| Vitamin C | 1,000 | mg | Calcium | 500 | mg |
| | | | (as amino acid chelate) | | |
| Vitamin D | 800 | IU | Iron | 20 | mg |
| | | | (as ferrous fumarate) | | |
| Vitamin E | 400 | IU | Iodine | 225 | mcg |
| Vitamin B1 | 25 | mg | Magnesium | 250 | mg |
| (thiamin) | | | (as amino acid chelate) | | |
| Vitamin B2 | 25 | mg | Zinc | 25 | mg |
| (riboflavin) | | | (as amino acid chelate) | | |
| Niacin | 150 | mg | Selenium | 25 | mcg |
| | | | (as Selenomethionine) | | |
| Nicotinamide | 150 | mg | Copper | 2 | mg |
| | | | (as amino acid chelate) | | |
| Vitamin B6 | 30 | mg | Manganese | 10 | mg |
| (pyridoxine) | | | (as amino acid chelate) | | |
| Vitamin B12 | 50 | mcg | Chromium picolinate | 1,000 | mcg |
| Folic acid | 400 | mcg | Potassium | 50 | mg |
| | | | (as amino acid chelate) | | |
| Biotin | 150 | mcg | Inositol | 100 | mg |
| Panthothenic acid | 150 | mg | | | |

The mixture was cooled to 30° C. with stirring, then poured into containers and cooled to room temperature. A gelatin-like system was obtained. Upon shaking at 30° C., the gel became liquid and after application on food surface it became solid again.

### Example 7

### Thixotropic Edible Canola Oil-Flavor System with Therapeutic Agents

The method of Example 6 was applied using 1 gram of cetyl alcohol instead of behenic acid. Similar results were obtained.

### Example 8

### Thickened Edible Chocolate-Flavored Primrose Oil Spread

Four grams of behenic acid were added to 96 grams of primrose oil and the mixture was stirred at 70° C. until the acid was completely dissolved in the oil. 8 grams cocoa powder, 10 grams sugar and 5 grams powdered milk were added and the mixture was cooled with stirring to 30° C., then poured into containers and cooled to room temperature. A tasty chocolate flavored thickened gel was obtained. It was used to coat different kinds of pastry products and was well accepted in tasting tests. 10 grams sugar could be substituted by 0.2 gram saccharine or 0.2 gram aspartame.

### Example 9

### Thixotropic Edible Chocolate-Flavored Primrose Oil Spread

The method of Example 8 was applied using 4 grams of beeswax-derived fatty alcohols instead of behenic acid. Results were similar.

### Example 10

### Popcorn

0.7 gram octacosanoic acid or, alternatively, 0.7 gram of beeswax-derived fatty alcohols and 0.5 gram salt were added to 18 grams of canola oil and the mixture was stirred at 80° C. until the acid was completely dissolved in the oil. The mixture was cooled with stirring to 30° C. Then, 100 grams of corn grains were added and mixed together with the oil-flavor system, until the grains were uniformly coated with oil, which was apparently solid. The mixture was filled into a paper bag and placed in the microwave for 4 minutes, at high level energy. At the end of this period, all grains were popped and the kernels were large and crispy.

### Example 11

### Microwave Popcorn-II

1.8 grams of behenic acid and 1.2 grams of palm oil flake were mixed together with 23 grams of canola oil at 80° C. Three grams of fine powdered salt were added to 71 grams of corn grains and the mixture was transferred into a microwave bag. The oil mixture was then added to the bag, at 70° C. The bag was sealed and cooled for solidification. The corn was then popped using a general procedure. All grains were popped and the kernels were large and crispy.

### Example 12

### Sweet Popcorn

The same method as in Example 10 was applied, but instead of salt, 5 grams of sugar were added to the oil system. Fifty grams of corn grains, coated with oil-sugar, were in a hot pot and allowed to pop (about 3 minutes). Large, off-white, crispy and sweet popcorn kernels were obtained.

### Example 13

### Flavored Pasta—Comparative Study

Italian-style pasta was prepared by mixing cooked pasta with the oil-flavor system of Example 2 or 3, in a ratio of 900 grams pasta and 100 grams oil-flavor system. The mixture was then divided to four 250 grams containers and 10 minutes afterwards, they were stored in the freezer at −4° C to −8° C. for 8 weeks. A control product, consisting of 900 grams pasta, 97 grams olive oil, 0.3 gram garlic extract, 0.5 gram salt and 0.2 gram of ground majoran, was prepared by mixing all ingredients in the same manner and for the same time, divided to 250 grams containers and stored in the freezer. At the end of storage, the pasta, treated with the oil-flavor system was uniformly coated with oil and only a minute amount of oil was found at the bottom of the container. In contrast, in each of the containers of the control product, the top layers of the pasta were virtually dry and about 15 grams oil was found at the bottom of the container. Likewise, most of the majoran was found at the bottom of the control containers.

### Example 14

### Pre-Fried Potato Chips

Four grams of octacosanoic acid, or alternatively, 4 grams of behenyl alcohol, were added to 1000 grams of soybean oil and the mixture was heated to 180° C. Then, 100 grams of fresh potato chips were added and fried for 2 minutes. The potato chips were removed from the oil and immediately stored at −20° C. The resulting product was similar in consistency to reference product, which was produced using conventional shortening. Yet the saturated oil content was similar to the original soybean oil.

### Example 15

Oil powder of 60% oil
Ingredients:

| | |
|---|---|
| Cellulose flavor Lc 200 | 31 grams |
| Any oil (olive) | 60 grams |
| Behenic acid | 5.4 grams |
| Palm flake | 3.6 grams |

Method:

Behenic acid and palm flake were melted in the oil (at about 80° C.). The cellulose fibers were heated to 70° C., added thereafter to the hot oil mixture and the final mixture was stirred until a complete absorbing was achieved. The mixture was then left at rest, at 70° C., for 8 hours, chilled to −20° C. and grained to maximum in a coffee grinder.

### Example 16

### Shortening

Ingredients:

92.5% RBD liquid canola oil

5% behenic acid or behenyl alcohol

1% emulsifier

2.5% fully hydrogenated vegetable oil

0.02% antioxidant mixture

Method:

All ingredients were mixed together at 70° C., to obtain a clear liquid. Mixture was than cooled down to 30° C. and poured to storage containers.

### Example 17

### Processing of Vegetable Oil Shortening, Vegetable Fat Shortening, Animal Fat Shortening, Fish Oil Shortening and Purable Shortening

Three grams of stearyl alcohol or behenyl alcohol were added to 1,000 grams of the oil or the fat. Seven grams of behenic acid were then added, and the mixture was heated to 80° C. and stirred until the alcohol and the acid were completely dissolved. The final mixture was then cooled to 65° C., poured into forma and chilled and/or freezed.

The solidifying agent concentration can be adjusted according to needed level of solidification.

### Example 18

### Ice-cream-I

Ingredients:

10% Shortening of example 16

20% Sugar

1% Emulsifier and stabilizer

69% Reconstituted milk

Edible flavors and colors

Method:

Pre-emulsion was prepared by warming all ingredients in the oil at 90° C. (Pasteurization). The mixture was homogenized using a high-pressure homogenizer and cooled to 4° C. with agitation, to allow re-crystallization. The re-crystallized mixture was chilled using scrape surface heat exchanger at −5° C., to yield a semi-solid product, which was transferred to final containers, which were, in turn, chilled at −20° C. to −40 C. for final solidification.

### Example 19

Ice-cream - II
Ingredients:

| | |
|---|---|
| Water | 64 grams |
| Sugar | 12 grams |
| Skim milk powder | 11.2 grams |
| Canola oil | 7.8 grams |
| Corn syrup | 3.7 grams |
| Stearyl alcohol | 0.26 grams |
| Emulsifier | 0.5 grams |
| Stabilizer | 0.15 grams |
| Color | As needed |
| Flavors | As needed |

Method:

The stearyl alcohol was dissolved in the oil, with stirring, at 70-75 C. The water was placed in a 1-liter beaker, and the corn syrup was added thereto. The dry ingredients were then added to the water phase and the mixture was stirred, using stirrer blades propeller, for at least half an hour at room temperature, to achieve a homogeneous phase. The aqueous mixture was then heated with stirring, using a water bath, to 75° C. and the warm oil solution was added thereto. Stirring was continued for 15 minutes and the mixture was then homogenized, using Ultra Turrax 1600 RMP, for 2.5 minutes. The mixture was cooled immediately thereafter, using an ice bath, with stirring, for about 15 minutes, kept at 4° C. for 4 hours for aging, and freezed at −6° C. in an ice cream freezer.

### Example 20

### Chocolate Cake

Ingredients:

15% Chocolate

15% Shortening of Example 16

20% Sugar

30% Eggs

20% Flour

Edible flavors and colors

Baking powder

Optional additives: milk; cream; cocoa; instant coffee; wine; brandy.

Method:

Eggs were separated to yolks and whites. Shortening was melted together with the chocolate. Egg whites were wiped with sugar to yield firm foam. Egg yolks were added with agitation to the shortening/chocolate mixture, then the flour was added with agitation. Foam was added with gentle agitation. The final mixture was baked at 200° C. for about 40-60 minutes.

### Example 21

### Poppy Cake

Ingredients:

20% Poppy

15% Shortening of Example 16

20% Sugar

25% Eggs

20% Flour

Edible flavors and colors

Baking powder

Optional additives: milk; cream; fruit juice; raisins; cocoa; instant coffee; wine; brandy.

Method:

Eggs were separated to yolks and whites. Shortening was melted together with egg yolks and liquid optional additives. Flour, poppy, baking powder and solid optional additives were mixed together. Egg whites were wiped with sugar to yield firm foam. The shortening/egg yolk mixture was added to the foam with gentle agitation. Solid mixture was added and the final mixture was placed in baking pan. The final mixture was baked at 200° C. for about 40-60 minutes.

### Example 22

Muffins (chocolate chips)
Ingredients:

| | |
|---|---|
| White of two | 80 grams |
| eggs | |
| Yolk of two | 40 grams |
| eggs | |
| Oil based | 85 grams |
| shortening | |
| Sugar | 25 grams |
| Sugar | 75 grams |
| Flour | 100 grams |
| Baking | 3.5 grams |
| powder | |
| Chocolate | 25 grams |
| chips | |
| Vanilla | |
| Total weight | 435 grams |

Method:

The eggs white was whipped with 75 grams sugar to yield a solid whip. The thixotropic oil-based shortening (of Example 16) was mixed in a bowl with the egg yolks and 25 grams sugar to yield a homogeneous paste, which was then mixed with the whip to produce butter. The flour was then added and the mixture was gently mixed. The chocolate chips and the vanilla were added thereafter to the butter mixture, which was then mixed and poured, optionally using a sprinkling bag, to a baking cone. The mixture was baked for about 45 minutes at 180° C., to yield 345 grams of the baked product.

### Example 23

Marble cake
Ingredients:
(for one form of 30 × 7 × 5 cm)

| | |
|---|---|
| Oil based shortening | 85 grams |
| Sugar | 100 grams |
| 2 eggs | 120 grams |
| Flour | 100 grams |
| Baking powder | 3.5 grams |
| Orange juice | 50 grams |
| Cocoa powder | 5 grams |
| Total weight | 465 grams |

Method:

The eggs were whipped with the sugar to produce a firm whip. The oil based shortening (of Example 16) was then gently mixed first with a small portion of the whip, to achieve a homogeneous paste, and then with the remaining whip to yield butter. Flour, baking powder and orange juice were added and the mixture was gently mixed. Three quarters of the mixture was poured into a baking form, and the remaining mixture was mixed with the cocoa powder and added thereafter to the form, using a sprinkling bag. The final mixture was mixed with a stick to achieve the marble shape and baked at 180° C. for 30 minutes.

### Example 24

Boullion
Ingredients:

| | |
|---|---|
| Mix of starch, dry vegetable, | 65 grams |
| salt, sugar and seasoning | |
| (powder) | |
| Canola oil | 29.5 grams |
| Behenic acid | 3.3 grams |
| (solidifying agent) | |
| Palm stearin | 2.2 grams |
| (solidifying agent) | |

Method:

The solidifying agents were added to the oil and the mixture was heated on a water bath. The powder was then added and the mixture was heated up to 85° C. The final mixture was then cooled to 70° C., transferred to a 25 ml forma and pressed in order to exclude a formation of an air pocket and to get smooth surface. The mixture was then cooled at ambient temperature or refrigerated.

### Example 25

### Margarine

Ingredients:

77.5% RBD liquid canola oil

16%Water

1% Mono-diglyceride emulsifier (45%)

0.5% Soy lecithin

0.5% Salt

0.4% Butter flavor

4% Steaiyl alcohol

0.1% Citric acid

Method:

Pre-emulsion was prepared by warming the oil to 60° C., adding the stearyl alcohol, emulsifier and lecithin and agitating, to obtain homogeneous solution. Water, salt and citric acid were then added to the mixture. The substance was chilled, rapidly mixed and transferred to jars. The resulting product was similar in consistency to a reference product, which was produced using conventional methods. Yet the saturated oil content was similar to the original canola oil. It was further found to be useful in spreading on bread and baking.

### Example 26

### Additional Margarine Compositions

Table 4 below presents additional compositions for the production of margarines having varying levels of thixotropy.

TABLE 4

| | Amount of ingredient per 100 gram | | |
|---|---|---|---|
| | 80% | 60% | 40% |
| Ingredient | Margarine | Margarine | Margarine |
| Water | 16.3 | 33.0 | 51.5 |
| Canola Oil | 73.0 | 54.0 | 37.0 |
| Behenic acid | 6.0 | 4.1 | 2.7 |
| Stearyl alcohol | 2.8 | 2.8 | 1.2 |
| Fibroline powder | — | 3.5 | 5.0 |
| Modified starch | — | 0.7 | 1.0 |
| Emulsifier | 1.0 | 0.7 | 0.5 |
| Stabilizer | 0.5 | 0.5 | 0.5 |
| Lecithin | — | 0.3 | 0.2 |
| Salt | 0.4 | 0.4 | 0.4 |
| Beta carotene color | As needed | As needed | As needed |
| Butter flavor | As needed | As needed | As needed |

Method:

The solidifying agents, behenic acid and stearyl alcohol, were added to a solution of the oil and the butter flavor and the mixture was heated to 70-75° C., with stirring. All the powder ingredients, except the salt, were added to the water and the aqueous mixture was stirred at room temperature for 15 minutes, using stirrer bladed propeller, to yield a homogeneous paste. The aqueous mixture was then heated gradually on a water bath to 60-65° C., while stirring was continued. The warm oil mixture was then added gradually to the water phase, with stirring, and the final mixture was emulsified, using stirrer blends knife for 60 seconds at 1500 RMP, at 60-65° C. The fine salt was then added and the mixture was further emulsified for 10 seconds at 100 RMP, cooled to 60° C., poured into containers and refrigerated.

### Example 27

### Thickened Sunflower Oil Composition

Four grams of stearyl alcohol were added to 96 grams of sunflower oil and the mixture was stirred at 60° C. until the alcohol was completely dissolved in the oil. The mixture was cooled to 30° C. with stirring, then poured into containers and cooled to room temperature. A gelatin-like system was obtained, with taste and smell similar to the original sunflower oil. Upon shaking at 30° C., the gel became liquid and after application on food surface it became solid again. The melting temperature of the resulting substance was 32°-36° C.

### Example 28

### Thickened Palm Oil and Other Oil Compositions

Three grams of behenyl alcohol (C-22) were added to 97 grams of palm oil (slip melting temperature −38° C.) and the mixture was stirred at 60° C. until the alcohol was completely dissolved in the oil. The mixture was cooled to 30° C. with stirring, then poured into containers and cooled to room temperature. A gelatin-like system was obtained, with taste and smell similar to the original palm oil. Upon shaking at 30° C., the gel became liquid and after application on food surface it became solid again. The melting temperature of the resulting substance was 42° C. The same process, using different fatty alcohols (C-16 to C-22) and different semi-solid and solid oils (palm oil, palm seed oil, coconut oil, sal oil, mango seed oil or butter oil) resulted in elevation of the respective melting temperature by at least 2° C.

### Example 29

### Preparation of Ice Cream Using a Thickened Oil Composition of Viscous Oil

A premix of milk powder (120 grams), sugar (200 grams), glucose syrup (30 grams), thickened oil composition of canola oil with 4% stearyl alcohol (120 grams), emulsifier (50 grams) and water to make one kilogram, was mixed, heated to 70° C., homogenized under pressure, pasteurized and cooled to 5° C. to allow crystallization. The mixture was further cooled at −20° C. to ⁰° C.

### Example 30

### Thickened Oil Composition of Unsaturated Oil

A premix of milk powder (20 grams), cocoa powder (60 grams), sugar (580 grams), thickened oil composition of soybean oil with 4% stearyl alcohol (300 grams), and lecithin (40 grams) was mixed, and heated thereafter to 60° C. with agitation for 24 hours. The mixture was then cooled to 40° C. and poured to a serving container.

### Example 31

### Modified Chocolate Substitute

A premix of milk powder (15 grams), cocoa powder (100 grams), sugar (580 grams), thickened oil composition of palm oil with 4% stearyl alcohol (300 grams) and lecithin (5 grams) was mixed, and heated thereafter to 60° C. with agitation for 24 hours. The mixture was then cooled to 40° C. and poured to tableting in mold.

### Example 32

Spreadable chocolate
Ingredients:
(for one form of 100 grams)

| | |
|---|---|
| Canola oil | 32.5 grams |
| (or any other vegetable Oil) | |
| Stearyl alcohol | 1 gram |
| Behenic acid | 2 grams |
| Fine powder sugar | 56 grams |
| Full fat soybean mill | 1.7 grams |
| Cocoa powder | 6.5 grams |
| Lecotaine | As needed |
| Aroma and flavors | As needed |

Method:

Stearyl alcohol and behenic acid were added to the oil and the mixture was heated to 80° C. Other powders were mixed together. The hot oil mixture was transferred to a jacketed heating/cooling balling mill (chocolate manufacturing equipment), the powder mixture was added gradually thereto, at 55-60° C., and the final mixture was minced to yield a smooth mass. The warm mixture was then poured into a container and cooled ambient.

### Example 33

### Foods in Which Modified Fat Substitute can be Incorporated (see Table 5)

TABLE 5

| Food Product | % Oil | Current Oil |
|---|---|---|
| Bread | 0.5-5 | Margarine or shortening |
| Cookies | 5-20 | Margarine or shortening |
| Muffin | 5-20 | Margarine or shortening |
| Biscuit | 5-40 | Margarine, shortening and/or |
| | | plant-derived semi-solid and solid fats |
| Cake | 5-20 | Margarine or shortening |
| Pizza | 5-30 | Margarine, shortening and/or |
| | | plant-derived semi-solid and solid fats |
| Pastry | 5-30 | Margarine |
| Doughnuts | 0.5-5 | Margarine or shortening |
| Halvah | 2-10 | Margarine, shortening and/or |
| | | plant-derived semi-solid and solid fats |
| Margarine-like spread | 10-80 | Shortening, hard-stock or plant-derived |
| | | semi-solid and solid fats |
| Chocolate spread | 10-50 | Margarine, shortening and/or |
| | | plant-derived semi-solid and solid fats |
| Chocolate substitute | 10-50 | Cocoa butter substitute and/or |
| | | plant-derived semi-solid and solid fats |
| Filling cream | 10-50 | Highly hydrogenated shortening and/or |
| | | plant-derived semi-solid and solid fats |
| Salad dressing | 5-60 | Vegetable oil, shortening and/or |
| | | plant-derived semi-solid and solid fats |
| Tehina (sesame spread) | 30-90 | Sesame oil |
| Fresh salads | 1-60 | Butter, animal fat, shortening and/or |
| | | plant-derived semi-solid and solid fats |
| Sauce | 1-60 | Butter, animal fat, vegetable oil, |
| | | shortening and/or plant-derived |
| | | semi-solid and solid fats |
| Ice cream | 1-30 | Butter, highly hydrogenated shortening |
| | | or hydrogenated palm oil |
| Ice cream coating | 10-50 | Hydrogenated palm or coconut oil |
| Cake coating | 10-50 | Hydrogenated palm or coconut oil |
| Milk and vegetable oil | 0.1-9 | Milk fat |
| Dairy products (yogurt, | 0.1-9 | Milk fat |
| flavored yogurt) | | |
| Cheese (soft, salted and cottage | 1-50 | Milk fat, animal-derived fat and/or |
| cheese, semi-solid cheese | | plant-derived semi-solid and solid fats |
| substitute) | | |
| “Low cholesterol meat | 1-30 | Shortening |
| products” (chopped meat) | | |
| Sausage | 1-60 | Animal fat, shortening and/or |
| | | plant-derived semi-solid and solid fats |
| Chopped meat products | 1-60 | Animal fat, shortening and/or |
| (hamburger, chicken, beef, fish, | | plant-derived semi-solid and solid fats |
| pork) | | |
| Vegetarian meat substitutes | 1-60 | Animal fat, shortening and/or |
| (chicken, beef, fish, pork-like) | | plant-derived semi-solid and solid fats |
| Soy-based meat substitutes | 1-30 | Animal fat, shortening and/or |
| | | plant-derived semi-solid and solid fats |
| Boullion | 1-30 | Animal fat, shortening and/or |
| | | plant-derived semi-solid and solid fats |
| Dry mix soup | 1-30 | Shortening, animal fat and/or |
| | | hydrogenated palm or coconut oil |

### Example 34

### Edible Olive Oil Spread

One gram of cetyl alcohol was added to 99 grams of olive oil and the mixture was stirred at 60° C. until the alcohol was completely dissolved in the oil. The hot mixture was then poured into containers and cooled to room temperature. A gelatin-like spread was obtained, with taste and smell similar to the original olive oil. GCMS analysis revealed that the lipid composition of the resulting spread was identical to the original oil.

### Example 35

### Seasoned Edible Canola Oil Spread

One gram of stearyl alcohol was added to 99 grams of canola oil and the mixture was stirred at 60° C. until the alcohol was completely dissolved in the oil. 0.2 gram of garlic extract, 0.4 gram salt and 0.2 gram ground majoran were added thereafter to the hot mixture and the mixture was cooled to 30° C. with stirring, then poured into containers and cooled to room temperature. A gelatin-like spread was obtained, with taste and smell similar to the original canola oil.

### Example 36

### Edible Fish Oil Spread

Four grams of beeswax-derived fatty alcohols were added to 96 grams of cod liver oil and the mixture was stirred at 60° C. until the alcohols were completely dissolved in the oil. 0.5 gram of lemon concentrate, 0.4 gram salt, 0.1 gram black pepper and 0.2 gram ground garlic were added to the hot mixture and the mixture was cooled to 30° C. with stirring, then poured into containers and cooled to room temperature. A gelatin-like spread was obtained. Analysis revealed that the lipid composition of the spread was identical to the original cod liver oil. The taste and smell of the resulting spread were distinctly different from the parent oil.

### Example 37

### Edible Fish Oil Spread—Acceptability Test

It is known in the art that at least 6 grams of cod liver oil is required in order to attain an effective blood lipid lowering effect. The purpose of this test was to assess the acceptability of the spread of Example 27, in comparison with the same amount of the oil (in its native liquid form). Ten volunteers were asked to apply 8 grams of the spread of Example 27 on one slice of bread and eat it. The same volunteers were asked to ingest the same amount of the parent cod liver oil and compare between the two options. All volunteers could apply an amount of 8 grams spread on one slice of bread and eat it with no difficulty. Their opinion was that the spread was tasty and that they could eat at least one more similar serving. In contrary, none of the volunteers was able to ingest the whole amount of liquid oil, due to repelling smell and taste.

### Example 38

### Edible Mineral Oil Spread

One gram of C-50 fatty alcohol (fatty alcohol having 50 carbon atoms) was added to 99 grams of pharmaceutical grade mineral oil and the mixture was stirred at 60° C. until the alcohol was completely dissolved in the oil. The hot mixture was then poured into containers and cooled to room temperature.

### Example 39

Vegetarian frankfurter with liquid oil
Ingredients:

| | |
|---|---|
| 59.8% | Water / ice (20 to 30% ice) |
| 11% | UNIT 1 (Supro EX 34 soya isolate) |
| 17% | Vegetable oil (sunflower or soy) |
| 2% | Behenic acid |
| 1% | Stearyl alcohol |
| 0.2% | UNIT 2 (functional protein blend) |
| 3% | UNIT 3 (spices and Salt) |

Method:

The water/ice mixture and UNIT 1 (Supro EX 34) were chopped to a fine gel. Shortening, prepared from behenic acid, stearyl alcohol and the oil, was added to the mixture slowly, and chopped until it was well emulsified. UNIT 2 was then added and the mixture was chopped until it became sticky. UNIT 3 was added thereafter and the final mixture was chopped until all spices were dispersed. The mixture was stuffed in peal casing dry, smoked and cooked at 80° C., to achieve internal degree of 75° C. The mixture was then washed, cooled, pealed and either packed in a vacuum pack or freezed.

### Example 40

Chocolate substitute bar
Ingredients:
(for 100 grams)

| | |
|---|---|
| Fine sugar powder | 50 grams |
| Cocoa powder | 15 grams |
| Palm oil or | 30 grams |
| Coconut oil | |
| Behenyl alcohol | 15 grams |
| Flavor lecithin | as needed |

Method:

The behenyl alcohol was melted in the oil at about 80° C. and the mixture was transferred to a jacketed heating/cooling balling mill (chocolate manufacturing equipment). Other powders were mixed, added gradually to the oil mixture at 55-60° C. and the final mixture was minced to yield smooth mass. The mixture was then poured to a chilled chocolate forma, at 50° C., chilled for solidification and stored at room temperature.

### Example 41

### Prevention of Oil Separation from Raw Tehina (Sesame Paste), Raw Peanut Butter and Raw Almond Paste

The concentration of the solidifying agent (stearyl alcohol, behenyl alcohol or behenic acid) was calculated to achieve a concentration range of 2-7% of the final product (depending the needed solidification level). Based on the calculation, a mixture of 40% solidifying agent in a typical oil was prepared by heating the mixture's components to 90° C., with stirring. The hot oil mixture was added to a pre-heated (70° C.) raw paste, with constant stirring. The paste was then cooled to 60° C. and transferred to a storage container.

### Example 42

Dressing for cooking comprising 40% canola oil
Ingredients (for 200 grams):

| | |
|---|---|
| Water | 100 grams |
| Emulsifier | 2 grams |
| Stabilizer | 1 gram |
| Canola oil | 78 grams |
| Behenic acid | 5.4 grams |
| Stearyl alcohol | 2.5 grams |
| Seasoning mix | 10 grams |
| Salt | 1 gram |

Method:

Behenic acid and stearyl alcohol were melted in the oil at 70-75° C. The emulsifier was dissolved in the water, using multi knife stirrer for at least 15 minutes at room temperature, to yield a homogeneous paste. The paste was heated gradually to 60-65° C. and mixed by stirrer blades propeller. The oil mixture was then added to the stirred water phase, the temperature was raised to 70° C., and the mixture was further stirred until the oil was completely absorbed. The final mixture was emulsified, using stirrer blades knife for 60 seconds at 1500 RMP, and cooled to 65° C. The fine salt was then added, the mixture was further emulsified for 10 seconds at 10,000 RPM and then refrigerated.

### Example 43

### Sprayed Oil Sheen for Producing Coated Aid Flavored/Colored Nuts, Crackers, Candies and Dried Fruits

A shortening was prepared from oil, 3-15% solidifying agent and antioxidant flavor or colorant as needed (see Example 16). The mixture was heated to 80° C., stirred, cooled to 70° C. and sprayed on the product through a nozzle.

### Example 44

Composition of bread comprising thickened canola oil

| | |
|---|---|
| Water | 150 grams |
| Skim milk | 15 grams |
| powder | |
| Canola oil | 30 grams |
| Behenic acid | 2 grams |
| Stearyl alcohol | 1 gram |
| Sugar | 15 grams |
| Salt | 5 grams |
| Wheat flour | 350 grams |
| Dry yeast | as needed |

### Example 45

Soybean isolate canola sausage
Ingredients:

| | |
|---|---|
| Soybean isolate porting | 15 grams |
| for sausage | |
| Canola oil | 15 grams |
| Behenic acid | 2 grams |
| Stearyl alcohol | 1 gram |
| Water | 67 grams |
| Colorants and flavors | as needed |

Method:

Water was added to the soybean isolate and the mixture was emulsified by cutter equipment. A mixture of the solidifying agents, the canola oil and colorant or flavor was added to the emulsion, and the final mixture was further emulsified, transferred to sausage coat and pasteurized at 75° C.

### Example 46

Solidified natural cream
Ingredients:

| | |
|---|---|
| Pasteurized caw cream | 100 grams |
| Stearyl or behenyl alcohol | 4 grams |

Method:

The cream was heated on a water bath and stirred with bladed propeller stirrer. At 60° C., the stearyl alcohol was added and heating was continued up to 80° C. The final mixture was then homogenized for 60 seconds, poured to container and refrigerated.

### Example 47

### Process for Canola Oil Cheese (Mozzarella Cheese)

Ingredients:

20% Vegetable oil (canola)

4% Stearyl or behenyl alcohol

22% Rennet casein

5% Skimmed milk powder

1.2% Trisodium citrate

0.2% Sodium hexametaphosphate

15% Salt

0.9% Citric acid

Water including condensate, up to 100%

Composition:

| | |
|---|---|
| Total solids | approx. 52% |
| Fat in dry matter | approx. 46% |

Method:

The solidifying agent was melted in the oil at about 75° C. Rennet casein and flavor were added, with stirring, and the mixture was heated to 60° C. The salts were added thereafter and the mixture was heated up to 75° C., with stirring, to yield homogeneous, smooth and elastic cheese. The final mixture was then transferred to modules, cooled to 5° C. and stored at 5

### Example 48

### Processed Cheese (20% Canola Oil)

Ingredients:

75% Non-fat rennet caw milk curd, 50% young, 40% mild, 10% Ripe

3% Emulsifying salt sodium citrate or mix of DD sodium phosphate or polyphosphate

20% Canola oil

2% Stearyl alcohol

Method:

A mantel heating vacuum pot with emulsifying knife system and direct steam injection for processed cheese cooking (e.g., Stephan pot, Germany) was used. The curd was grounded, mixed with the emulsifying salt and put in a cooking pot. Stearyl alcohol was melted in the canola oil and the oil mixture was added to the grounded curd. The final mixture was heated to 75-76° C., emulsified for 2 minutes, poured immediately to forma and refrigerated.

One gram of palm flake can be added to the mixture to yield higher solidification level.

### Example 49

40% Fat light butter
Ingredients:

| | |
|---|---|
| Butter | 50 grams |
| Water | 46 grams |
| Behenic acid | 28 grams |
| Stearyl alcohol | 12 grams |
| Emulsifier | 10 grams |

Method:

Behenic acid and stearyl alcohol were melted in the butter at about 80° C. The emulsifier was dissolved in the water by mixing with stirrer bladed propeller for at least 15 minutes at room temp, and the water phase was then heated gradually. The hot butter mixture was added to the stirred water phase, the temperature was raised to 75° C. and the mixture was stirred until the oil was completely absorbed. The final mixture was then emulsified by stirrer blades knife for 60 seconds at 1500 RPM, cooled to 65° C., poured to container and cooled.

### Example 50

10% Canola oil quark cheese
Ingredients:

| | |
|---|---|
| Skim quark (15% dry matter) | 89 grams |
| Canola oil | 10 grams |
| Stearyl alcohol | 0.7 grams |
| Palm flake | 0.3 grams |

Method:

The solidifying agent was melted in the canola oil at about 78° C. The quark was heated to 70° C., with stirring, and the oil mixture was added thereto. The final mixture was then homogenized using Ultra Turret 16,000 RPM for 30 seconds, cooled to 68° C., poured to container and refrigerated.

### Example 51

Solidified 38% natural cream - plastic cream
Ingredients

| | |
|---|---|
| Pasteurized caw cream 38% | 96 grams |
| Stearyl or behenyl alcohol | 46 grams |

Method:

The cream was heated on a water bath to 60° C. and stirred with bladed propeller stirrer. Stearyl alcohol was added thereto and heating was continued up to 80° C. The final mixture was then homogenized for 60 seconds with stirring, poured to container and refrigerated.

### Example 52

### 5% Canola Oil Milk (Filled Milk) and Yogurt

Ingredients:

94.8% Skim caw milk

5% Canola oil

0.2% Stearyl alcohol

Method:

Stearyl alcohol was melted in the canola oil at about 60° C. The milk was heated to 70° C. and stirred with bladed propeller stirrer. The hot oil mixture was added thereto and heating was continued up to 80° C. The final mixture was then homogenized using Ultra Turret at 1600 RPM for 60 seconds, and further homogenized thereafter at 2000 and 500 PSR (pressure type homogenized). The mixture was cooled immediately thereafter and refrigerated.

Yogurt can prepared by adding culture to the mixture and incubate for acidification.

### Example 53

### 15% Canola Oil Cream (Filled Cream)—Liquid or Acidified

Ingredients:

84.5% Skim caw milk

15% Canola oil

0.5% Stearyl alcohol

Method:

Stearyl alcohol was melted in the canola oil at about 60° C. The milk was heated to 70° C. and stirred with bladed propeller stirrer. The hot oil mixture was added thereto and heating was continued up to 80° C. The final mixture was then homogenized using Ultra Turret at 1600 RPM for 60 seconds and further homogenized thereafter at 2000 and 500 PSR (pressure type homogenized). The mixture was cooled immediately thereafter and refrigerated.

Yogurt can be prepared by adding culture to the mixture and incubate for acidification.

### Example 54

### Cocoa Butter Substitute

Cocoa butter substitute can be prepared from lauric oils such as coconut oil, palm kernel oil or any hydrogenated lauric oil.

Ingredients:

90 grams oil

10 grams behenyl alcohol or behenic acid

0-10 grams palm flake, depending the needed level of solidification.

Method:

Behenyl alcohol or behenic acid and, optionally, palm flake, were added to the oil. The mixture was heated with stirring up to 80° C., cooled to 65° C., poured to forma and chilled in either a refrigerator or a freeze.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims. All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention.

What is claimed is:

1. An edible product comprising a base edible product or material and a thickened oil composition, said thickened oil composition including a combination of at least one edible oil and at least one edible solidifying agent, said edible solidifying agent being for converting said edible oil into said thickened oil composition.

2. The edible product of claim 1, wherein said base edible product or material is selected from the group consisting of a food and an edible medicament.

3. The edible product of claim 1, wherein said thickened oil composition is externally applied onto said base edible product or material.

4. The edible product of claim 1, wherein said thickened oil composition is mixed with said base edible product or material.

5. The edible product of claim 1, wherein said thickened oil composition is a thixotropic composition, said at least one edible solidifying agent converting said edible oil into said thixotropic composition, wherein said thixotropic composition exhibits a substantially semi-solid state upon rest at a temperature above the melting temperature of said at least one edible solidifying agent and a substantially liquid form upon being mechanically disturbed at said temperature.

6. The edible product of claims 1, wherein said at least one edible solidifying agent includes an edible long chain fatty acid and/or an edible solidifying agent having a molecular weight of at least 200 Da and at least one hydroxyl group.

7. The edible product of claim 1, wherein said at least one edible oil is present at a concentration ranging from 50 to 99 percent by weight of said thickened oil composition.

8. The edible product of claim 6, wherein said edible long chain fatty acid has 18 to 50 carbon atoms in its hydrocarbon chain.

9. The edible product of claim 6, wherein said edible long chain fatty acid has at least one double bond in its hydrocarbon chain.

10. The edible product of claim 6, wherein said edible long chain fatty acid has at least one branched alkyl group side chain.

11. The edible product of claim 1, wherein said at least one edible oil comprises an unsaturated oil.

12. The edible product of claim 1, wherein said at least one edible oil comprises a major portion of an unsaturated oil.

13. The edible product of claim 11, wherein said unsaturated oil is present at a concentration range of from 70 to 99.8 percent by weight.

14. The edible product of claim 11, wherein said unsaturated oil comprises a major portion of a poly-unsaturated oil.

15. The edible product of claim 1, wherein said at least one edible oil includes at least 6 percent by weight of an oil selected from the group consisting of omega-3 oil, omega-6 oil and a mixture thereof.

16. The edible product of claim 1, wherein said at least one edible oil is derived from a vegetable, a terrestrial animal, a marine origin or a combination thereof.

17. The edible product of claim 1, wherein said at least one edible oil is selected from the group consisting of palm seed oil, sal oil, mango seed oil, butter oil, cocoa oil, olive oil, soybean oil, canola oil, rapeseed oil, cottonseed oil, coconut oil, palm oil, sesame oil, sunflower oil, borage seed oil, syzigium aromaticum oil, hempseed oil, herring oil, cod-liver oil, salmon oil, corn oil, flaxseed oil, wheat germ oil, evening primrose oil, castor oil, linen oil, matthiola incana oil, garlic oil, shark oil, hypericum oil, sweet basil oil, mineral oil, silicone oil, highly saturated oils from animal sources and a mixture thereof.

18. The edible product of claim 1, further comprising at least one edible excipient, selected from the group consisting of water, a surfactant, an emulsifier, a monoglyceride, a diglyceride, a triglyceride, a flavoring agent, a colorant agent and an odorant agent.

19. The edible product of claim 15, wherein said flavoring agent is selected from the group consisting of sugar, salt, pepper and cumin.

20. The edible product of claim 1, further comprising at least one therapeutic agent in an amount up to 20 times the recommended daily dose per serving unit.

21. The edible product of claim 20, wherein said at least one therapeutic agent is selected from the group consisting of vitamin A, vitamin C, vitamin D, vitamin E, vitamin B1 (thiamin), vitamin B2 (riboflavin), niacine, nicotinamide, vitamin B6 (pyridoxine), vitamin B12, folic acid, biotin, panthothenic acid, inorganic salts or organometallic complexes of sodium, calcium, iron, iodine, magnesium, zinc, selenium, copper, manganese, chromium and vanadium, powder or extracts of aloe vera, anise, balm, baneberry, basil, thyme, bearberry, bloodroot, cedar, chamomile, chicory, cloves, echinacea, fennel, feverfew, garlic, ginseng, horse nettle, ivy, juniper, lavender, horse nettle, onion, parsley, peppermint, rosemary, sage, saw palmetto berries, St. John's wort, senna, thyme, valerian, yarrow and a mixture thereof.

22. The edible product of claim 6, wherein said edible solidifying agent having the molecular weight of at least 200 Da and at least one hydroxyl group comprises a fatty alcohol derivative having at least 15 carbon atoms in its hydrocarbon chain.

23. The edible product of claim 22, wherein said fatty alcohol derivative has up to about 50 carbon atoms in its hydrocarbon chain.

24. The edible product of claim 23, wherein said fatty alcohol derivative comprises an edible di-alcohol of formula A: 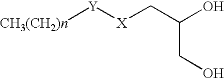
wherein, n=8−48;
X═CHR, O, or NH;
Y═CHR; or C═O; and
R═H or alkyl.

25. The edible product of claim 23, wherein said fatty alcohol derivative comprises an edible di-alcohol of formula B: 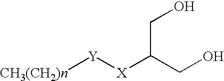
wherein, n=8-48;
X═CHR, O, or NH;
Y═CHR; or C═O; and
R═H or alkyl.

26. The edible product of claim 1, further comprising a medicament.

27. The edible product of claim 1, wherein said at least one solidifying agent comprises a wax substance, or a mixture of fatty alcohols derived from beeswax, a majority of said fatty alcohols having at least 20 carbon atoms in their hydrocarbon chains.

28. A process of producing an edible product, the process comprising:
(i) preparing a thickened oil composition by blending a major portion of at least one edible oil with at least one edible solidifying agent;
(ii) applying said thickened oil composition in substantially liquid form to a base edible product or material; and
(iii) maintaining said thickened oil composition in substantially liquid form in contact with said base edible product or material for a time sufficient to permit at least a portion of said thickened oil composition to adhere to said base edible product or material.

29. The process of claim 28, wherein step (ii) comprises applying said thickened oil composition to surfaces of said base food product or material so as to achieve a substantially uniform distribution of said thickened oil composition thereupon.

30. The process of claim 28, further comprising, prior to step (ii), adding to said thickened oil composition at least one edible excipient selected from the group consisting of water, a surfactant, an emulsifier, a monoglyceride, a diglyceride, a triglyceride, a flavoring agent, a colorant agent, an odorant agent, a herbal substance, a therapeutic agent, a medicament and a mixture thereof.

31. The process of claim 28, wherein said edible base product or material is selected from the group consisting of a food and an edible medicament.

32. The process of claim 28, wherein said thickened oil composition is externally applied onto said base edible product or material.

33. The process of claim 28, wherein said thickened oil composition is mixed with said base edible product or material.

34. The process of claim 28, wherein said thickened oil composition is a thixotropic composition, said at least one edible solidifying agent converting said edible oil into said thixotropic composition, wherein said thixotropic composition exhibits, at a temperature above the melting temperature of said at least one edible solidifying agent, a substantially semi-solid state upon rest and a substantially liquid form upon being mechanically disturbed at said temperature.

35. The process of claim 28, wherein said at least one edible solidifying agent is an edible long chain fatty acid and/or an edible solidifying agent having a molecular weight of at least 200 Da and at least one hydroxyl group.

36. The process of claim 28, wherein said at least one edible oil is present at a concentration ranging from 50 to 99 percent by weight of said thickened oil composition.

37. The process of claim 35, wherein said edible long chain fatty acid has 18 to 50 carbon atoms in its hydrocarbon chain.

38. The process of claim 35, wherein said edible long chain fatty acid has at least one double bond in its hydrocarbon chain.

39. The process of claim 35, wherein said edible long chain fatty acid has at least one branched alkyl group side chain.

40. The process of claim 28, wherein said at least one edible oil comprises an unsaturated oil.

41. The process of claim 28, wherein said at least one edible oil comprises a major portion of an unsaturated oil.

42. The process of claim 40, wherein said unsaturated oil is present at a concentration range of from 70 to 99.8 percent by weight.

43. The process of claim 40, wherein said unsaturated oil comprises a major portion of a poly-unsaturated oil.

44. The process of claim 28, wherein said at least one edible oil includes at least 6 percent by weight of an oil selected from the group consisting of omega-3 oil, omega-6 oil and a mixture thereof.

45. The process of claim 28, wherein said at least one edible oil is derived from a vegetable, a terrestrial animal, a marine origin or a combination thereof.

46. The process of claim 28, wherein said at least one edible oil is selected from the group consisting of palm seed oil, sal oil, mango seed oil, butter oil, cocoa oil, olive oil, soybean oil, canola oil, rapeseed oil, cottonseed oil, coconut oil, palm oil, sesame oil, sunflower oil, borage seed oil, syzigium aromaticum oil, hempseed oil, herring oil, cod-liver oil, salmon oil, corn oil, flaxseed oil, wheat germ oil, evening primrose oil, castor oil, linen oil, matthiola incana oil, garlic oil, shark oil, hypericum oil, sweet basil oil, mineral oil, silicone oil, highly saturated oils from animal sources and a mixture thereof.

47. The process of claim 28, wherein said edible product further comprising at least one edible excipient, selected from the group consisting of water, a surfactant, an emulsifier, a monoglyceride, a diglyceride, a triglyceride, a flavoring agent, a colorant agent and an odorant agent.

48. The process of claim 47, wherein said flavoring agent is selected from the group consisting of sugar, salt, pepper and cumin.

49. The process of claim 28, further comprising adding at least one therapeutic agent in an amount up to 20 times the recommended daily dose per serving unit.

50. The process of claim 49, wherein said at least one therapeutic agent is selected from the group consisting of vitamin A, vitamin C, vitamin D, vitamin E, vitamin B1 (thiamin), vitamin B2 (riboflavin), niacine, nicotinamide, vitamin B6 (pyridoxine), vitamin B12, folic acid, biotin, panthothenic acid, inorganic salts or organometallic complexes of sodium, calcium, iron, iodine, magnesium, zinc, selenium, copper, manganese, chromium and vanadium, powder or extracts of aloe vera, anise, balm, banebeny, basil, thyme, bearberry, bloodroot, cedar, chamomile, chicory, cloves, echinacea, fennel, feverfew, garlic, ginseng, horse nettle, ivy, juniper, lavender, horse nettle, onion, parsley, peppermint, rosemary, sage, saw palmetto berries, St John's wort, senna, thyme, valerian, yarrow and a mixture thereof.

51. The process of claim 35, wherein said edible solidifying agent having the molecular weight of at least 200 Da and at least one hydroxyl group comprises a fatty alcohol derivative having at least 15 carbon atoms in its hydrocarbon chain.

52. The process of claim 51, wherein said fatty alcohol derivative has up to about 50 carbon atoms in its hydrocarbon chain.

53. The process of claim 52, wherein said fatty alcohol derivative comprises an edible di-alcohol of formula A; 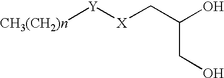
wherein, n=8−48;
X═CHR, O, or NH;
Y═CHR; or C═O; and
R═H or alkyl.

54. The process of claim 52, wherein said fatty alcohol derivative comprises an edible di-alcohol of formula B: 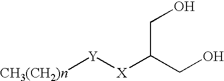
wherein, n=8−48;
X═CHR, O, or NH;
Y═CHR; or C═O; and
R═H or alkyl.

55. The process of claim 28, further comprising addition of a medicament.

56. The process of claim 28, wherein said at least one solidifying agent comprises a wax substance, or a mixture of fatty alcohols derived from a wax substance, a majority of said fatty alcohols having at least 20 carbon atoms in their hydrocarbon chains.

57. A food product comprising a base food product or material and a thickened oil composition, said thickened oil composition including at least one edible oil and at least one edible solidifying agent, said edible solidifying agent being for converting said edible oil into said thickened oil composition.

58. The food product of claim 57, wherein said thickened oil composition is a thixotropic composition, said at least one edible solidifying agent converting said edible oil into said thixotropic composition, wherein, at a temperature above the melting temperature of said at least one edible solidifying agent, said thixotropic composition exhibits a substantially semi-solid state upon rest and a substantially liquid form upon being mechanically disturbed at said temperature.

59. The food product of claim 57, wherein said at least one edible solidifying agent is an edible long chain fatty acid and/or an edible solidifying agent having a molecular weight of at least 200 Da and at least one hydroxyl group.

60. The food product of claim 57, wherein said at least one edible oil is present at a concentration ranging from 70 to 99 percent by weight and said edible solidifying agent is present at a concentration ranging from 1 to 30 percent by weight.

61. The food product of claim 59, wherein said edible long chain fatty acid has 18 to 50 carbon atoms in its hydrocarbon chain.

62. The food product of claim 59, wherein said edible long chain fatty acid has at least one double bond in its hydrocarbon chain.

63. The food product of claim 59, wherein said edible long chain fatty acid has at least one alkyl group side chain.

64. The food product of claim 59, wherein said edible solidifying agent having the molecular weight of at least 200 Da and at least one hydroxyl group comprises a fatty alcohol derivative, having at least 15 carbon atoms in its hydrocarbon chain.

65. The food product of claim 64, wherein said fatty alcohol derivative has up to about 50 carbon atoms in its hydrocarbon chain.

66. The food product of claim 65, wherein said fatty alcohol derivative comprises an edible di-alcohol of formula A: 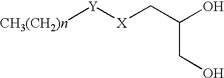
wherein, n=8−48;
X═CHR, O, or NH;
Y═CHR; or C═O; and
R═H or alkyl.

67. The food product of claim 65, wherein said fatty alcohol derivative comprises an edible di-alcohol of formula B: 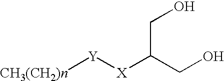
wherein, n=8−48;
X═CHR, O, or NH;
Y═CHR; or C═O; and
R═H or alkyl.

68. The food product of claim 57, wherein said at least one edible solidifying agent comprises a wax substance, or a mixture of fatty alcohols derived from a wax substance, a majority of said fatty alcohols having at least 20 carbon atoms in their hydrocarbon chains.

69. The food product of claim 57, wherein said at least one edible oil comprises an unsaturated oil.

70. The food product of claim 57, wherein said at least one edible oil comprises a major portion of an unsaturated oil.

71. The food product of claim 69, wherein said unsaturated oil is present at a concentration range of from 70 to 99.8 percent by weight.

72. The food product of claim 69, wherein said unsaturated oil comprises a major portion of poly-unsaturated oils.

73. The food product of claim 57, wherein said at least one edible oil includes at least 6 percent by weight of an oil selected from the group consisting of omega-3 oil, omega-6 oil and a mixture thereof.

74. The food product of claim 69, wherein said unsaturated oil is of vegetable or marine origin.

75. The food product of claim 74, wherein said unsaturated oil is selected from the group consisting of olive oil, soybean oil, canola oil, rapeseed oil, cottonseed oil, coconut oil, palm oil, sesame oil, sunflower oil, borage seed oil, syzigium aromaticum oil, hempseed oil, herring oil, cod-liver oil, salmon oil, corn oil, flaxseed oil, wheat germ oil, evening primrose oil and a mixture thereof.

76. The food product of claim 57, wherein said thickened oil composition further comprising at least one food excipient selected from the group consisting of water, a surfactant, an emulsifier, a diglyceride, a triglyceride, a flavoring agent, a colorant agent, an odorant agent and a therapeutic agent.

77. The food product of claim 76, wherein said flavoring agent is selected from the group consisting of sugar, salt, pepper and cumin.

78. The food product of claim 57, wherein said thickened oil composition further comprising at least one therapeutic agent present in an amount of up to 20 times the recommended daily dose per serving unit.

79. The food product of claim 78, wherein said at least one therapeutic agent is selected from the group consisting of vitamin A, vitamin C, vitamin D, vitamin E, vitamin B1 (thiamin), vitamin B2 (riboflavin), niacine, nicotinamide, vitamin B6 (pyridoxine), vitamin B12, folic acid, biotin, panthothenic acid, inorganic salts or organometallic complexes of sodium, calcium, iron, iodine, magnesium, zinc, selenium, copper, manganese, chromium and vanadium, powder or extracts of aloe vera, anise, balm, baneberry, basil, thyme, bearberry, bloodroot, cedar, chamomile, chicory, cloves, echinacea, fennel, feverfew, garlic, ginseng, horse nettle, ivy, juniper, lavender, horse nettle, onion, parsley, peppermint, rosemary, sage, saw palmetto berries, St. John's wort, senna, thyme, valerian, yarrow and a mixture thereof.

80. The food product of claim 57, wherein said thickened oil composition further comprising a medicament.

81. The food product of claim 57, wherein said base food product or material is selected from the group consisting of a fresh, processed, or pre-cooked starch, carbohydrate, protein, fat, potato, wheat, corn, barley, oat, rye, millet, vegetable, fruit, dried fruit, tuber, nut, bean, seed, rice, fish, egg, milk, cheese, ice cream, meat, sausage, meat substitute and poultry.

82. The food product of claim 57, wherein said base food product or material is selected from the group consisting of a fresh, processed, or pre-cooked popcorn, pastry, bread, baked good, pasta, pizza, batter, bagel, cake, biscuit, cracker, potato chip, tortilla, snack food, confection, shortening, margarine, processed butter, salad dressing, meat sauce, fish sauce, garlic sauce, mushroom sauce, mustard, chick pee spread, sesame spread, chocolate spread, peanut butter, almond butter, seed butter, vegetable powder comestible, fruit powder comestible, cocoa powder and soup pre-mixed powder comestible.

83. The food product of claim 57, wherein said base food product or material is a pre-fried food material selected from the group consisting of popcorn, potato chip, tortilla, nut, peanut, almond, banana chip, seed, vegetable, fish, meat and fruit.

84. The food product of claim 57, wherein said thickened oil composition comprises a portion ranging from 1 to 95 percent by weight of the food product.

85. The food product of claim 57, wherein said thickened oil composition is externally applied onto said base food product or material.

86. The food product of claim 57, wherein said thickened oil composition is mixed with said base food product or material.

87. A process of a food product, the process comprising:
(i) preparing a thickened oil composition by blending a major portion of at least one edible oil with at least one edible solidifying agent;
(ii) applying said thickened oil composition in substantially liquid form to a base food product or material; and
(iii) maintaining said thickened oil composition in substantially liquid form in contact with said base food product or material for a time sufficient to permit at least a portion of said thickened oil composition to adhere to said base food product or material.

88. The process of claim 87, wherein step (ii) comprises applying said thickened oil composition to surfaces of said base food product or material, so as to achieve a substantially uniform distribution of said thickened oil composition thereupon.

89. The process of claim 87, further comprising, prior to step (ii), adding to said thickened oil composition at least one food excipient selected from the group consisting of water, a surfactant, an emulsifier, a monoglyceride, a diglyceride, a triglyceride, a flavoring agent, a colorant agent, an odorant agent, herbal substance, a therapeutic agent, a medicament and a mixture thereof.

90. The process of claim 87, wherein said thickened oil composition is a thixotropic composition, said at least one edible solidifying agent converting said edible oil into said thixotropic composition, wherein, at a temperature above the melting temperature of said at least one edible solidifying agent, said thixotropic composition exhibits a substantially semi-solid state upon rest and a substantially liquid form upon being mechanically disturbed at said temperature.

91. The process of claim 87, wherein said at least one edible solidifying agent is an edible long chain fatty acid and/or an edible solidifying agent having a molecular weight of at least 200 Da and at least one hydroxy group.

92. The process of 87, wherein said at least one edible oil is present at a concentration ranging from 70 to 99 percent by weight and said edible solidifying agent is present at a concentration ranging from 1 to 30 percent by weight.

93. The process of claim 91, wherein said edible long chain fatty acid has 18 to 50 carbon atoms in its hydrocarbon chain.

94. The process of claim 91, wherein said edible long chain fatty acid has at least one double bond in its hydrocarbon chain.

95. The process of claim 91, wherein said edible long chain fatty acid has at least one alkyl group side chain.

96. The process of claim 91, wherein said edible solidifying agent having the molecular weight of at least 200 Da and at least one hydroxyl group comprises a fatty alcohol derivative, having at least 15 carbon atoms in its hydrocarbon chain.

97. The process of claim 96, wherein said fatty alcohol derivative has up to about 50 carbon atoms in its hydrocarbon chain.

98. The process of claim 97, wherein said fatty alcohol derivative comprises an edible di-alcohol of formula A: 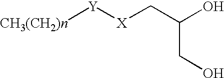
wherein, n=8−48;
X═CHR, O, or NH;
Y═CHR; or C═O; and
R═H or ally.

99. The process of claim 97, wherein said fatty alcohol derivative comprises an edible di-alcohol of formula B: 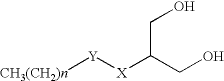
wherein, n=8−48;
X═CHR, O, or NH;
Y═CHR; or C═O; and
R═H or alkyl.

100. The process of claim 87, wherein said at least one edible solidifying agent comprises a wax substance, or a mixture of fatty alcohols derived from a wax substance, a majority of said fatty alcohols having at least 20 carbon atoms in their hydrocarbon chains.

101. The process of claim 87, wherein said at least one edible oil comprises an unsaturated oil.

102. The process of claim 87, wherein said at least one edible oil comprises a major portion of an unsaturated oil.

103. The process of claim 101, wherein said unsaturated oil is present at a concentration range of from 70 to 99.8 percent by weight.

104. The process of claim 101, wherein said unsaturated oil comprises a major portion of poly-unsaturated oils.

105. The process of claim 87, wherein said at least one edible oil includes at least 6 percent by weight of an oil selected from the group consisting of omega-3 oil, omega-6 oil and a mixture thereof.

106. The process of claim 101, wherein said unsaturated oil is of vegetable or marine origin.

107. The process of claim 106, wherein said unsaturated oil is selected from the group consisting of olive oil, soybean oil, canola oil, rapeseed oil, cottonseed oil, coconut oil, palm oil, sesame oil, sunflower oil, borage seed oil, syzigium aromaticum oil, hempseed oil, herring oil cod-liver oil, salmon oil, corn oil, flaxseed oil, wheat germ oil, evening primrose oil and a mixture thereof.

108. The process of claim 87, wherein said thickened oil composition further comprises at least one food excipient selected from the group consisting of water, a surfactant, an emulsifier, a diglyceride, a triglyceride, a flavoring agent, a colorant agent, an odorant agent and a therapeutic agent.

109. The process of claim 108, wherein said flavoring agent is selected from the group consisting of sugar, salt, pepper and cumin.

110. The process of claim 87, wherein said thickened oil composition further comprises at least one therapeutic agent present in an amount of up to 20 times the recommended daily dose per serving unit.

111. The process of claim 110, wherein said at least one therapeutic agent is selected from the group consisting of vitamin A, vitamin C, vitamin D, vitamin E, vitamin B1 (thiamin), vitamin B2 (riboflavin), niacine, nicotinamide, vitamin B6 (pyridoxine), vitamin B12, folic acid, biotin, panthothenic acid, inorganic salts or organometallic complexes of sodium, calcium, iron, iodine, magnesium, zinc, selenium, copper, manganese, chromium and vanadium, powder or extracts of aloe vera, anise, balm, baneberry, basil, thyme, bearberry, bloodroot, cedar, chamomile, chicory, cloves, echinacea, fennel, feverfew, garlic, ginseng, horse nettle, ivy, juniper, lavender, horse nettle, onion, parsley, peppermint, rosemary, sage, saw palmetto berries, St. John's wort, senna, thyme, valerian, yarrow and a mixture thereof.

112. The process of claim 87, wherein said thickened oil composition further comprises a medicament.

113. The process of claim 87, wherein said base food product or material comprises a food material selected from the group consisting of a fresh, processed, or pre-cooked starch, carbohydrate, protein, fat, potato, wheat, corn, barley, oat, rye, millet, vegetable, fruit, dried fruit, tuber, nut, bean, seed, rice, fish, egg, milk, cheese, ice cream, meat, sausage, meat substitute and poultry.

114. The process of claim 87, wherein said base food product or material comprises a food material selected from the group consisting of a fresh, pro, or pre-cooked popcorn, pastry, bread, baked good, pasta, pizza, batter, bagel, cake, biscuit, cracker, potato chip, tortilla, snack food, confection, shortening, margarine, processed butter, salad dressing, meat sauce, fish sauce, garlic sauce, mushroom sauce, mustard, chick pee spread, sesame spread, chocolate spread, peanut butter, almond butter, seed butter, vegetable powder comestible, fruit powder comestible, cocoa powder and soup pre-mixed powder comestible.

115. The process of claim 87, wherein said base food product or material is a pre-fried food material selected from the group consisting of popcorn, potato chip, tortilla, nut, peanut, almond, banana chip, seed, vegetable, fish, meat and fruit.

116. The process of claim 87, wherein said thickened oil composition comprises a portion ranging from 1 to 95 percent by weight of the food base product.

117. The process of claim 87, wherein said thickened oil composition is externally applied onto said base food product or material.

118. The process of claim 87, wherein said thickened oil composition i's mixed with said base food product or material.

119. A thickened oil composition comprising at least one edible oil and at least one edible solidifying agent, said edible solidifying agent being for converting said edible oil into the thickened oil composition.

120. The thickened oil composition of claim 119, wherein said at least one edible solidifying agent converting said edible oil into a thixotropic composition, whereas, at a temperature above the melting temperature of said at least one edible solidifying agent, said thixotropic composition exhibits a substantially semi-solid state upon rest and a substantially liquid form upon being mechanically disturbed at said temperature.

121. The thickened oil composition of claim 119, wherein said at least one edible solidifying agent is an edible long chain fatty acid and/or an edible solidifying agent having a molecular weight of at least 200 Da and at least one hydroxyl group.

122. The thickened oil composition of claim 119, wherein said at least one edible oil is present at a concentration ranging from 70 to 99 percent by weight and said edible solidifying agent is present at a concentration ranging from 1 to 30 percent by weight.

123. The thickened oil composition of claim 121, wherein said edible long chain fatty acid has 18 to 50 carbon atoms in its hydrocarbon chain.

124. The thickened oil composition of claim 121, wherein said edible long chain fatty acid has at least one double bond in its hydrocarbon chain.

125. The food product of claim 121, wherein said edible long chain fatty acid has at least one alkyl group side chain.

126. The food product of claim 121, wherein said edible solidifying agent having the molecular weight of at least 200 Da and at least one hydroxyl group comprises a fatty alcohol derivative, having at least 15 carbon atoms in its hydrocarbon chain.

127. The food product of claim 126, wherein said fatty alcohol derivative has up to about 50 carbon atoms in its hydrocarbon chain.

128. The food product of claim 127, wherein said fatty alcohol derivative comprises an edible di-alcohol of formula A: 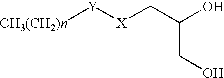
wherein, n=8−48;
X═CHR, O, or NH;
Y═CHR; or C═O; and
R═H or alkyl.

129. The food product of claim 127, wherein said fatty alcohol derivative comprises an edible di-alcohol of formula B: 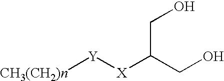
wherein, n=8−48;
X═CHR, O, or NH;
Y═CHR; or C═O; and
R═H or alkyl.

130. The thickened oil composition of claim 119, wherein said at least one edible solidifying agent comprises a wax substance, or a mixture of fatty alcohols derived from a wax substance, a majority of said fatty alcohols having at least 20 carbon atoms in their hydrocarbon chains.

131. The thickened oil composition of claim 119, wherein said at least one edible oil comprises an unsaturated oil.

132. The thickened oil composition of claim 119, wherein said at least one edible oil comprises a major portion of an unsaturated oil.

133. The thickened oil composition of claim 131, wherein said unsaturated oil is present at a concentration range of from 70 to 99.8 percent by weight.

134. The thickened oil composition of claim 131, wherein said unsaturated oil comprises a major portion of poly-unsaturated oils.

135. The thickened oil composition of claim 119, wherein said at least one edible oil includes at least 6 percent by weight of an oil selected from the group consisting of omega-3 oil, omega-6 oil and a mixture thereof.

136. The thickened oil composition of claim 131, wherein said unsaturated oil is of vegetable or marine origin.

137. The thickened oil composition of claim 136, wherein said unsaturated oil is selected from the group consisting of olive oil, soybean oil, canola oil, rapeseed oil, cottonseed oil, coconut oil, palm oil, sesame oil, sunflower oil, borage seed oil, syzigium aromaticum oil, hempseed oil, herring oil, cod-liver oil, salmon oil, corn oil, flaxseed oil, wheat germ oil, evening primrose oil and a mixture thereof.

138. The thickened oil composition of claim 119, further comprising at least one edible excipient selected from the group consisting of water, a surfactant, an emulsifier, a diglyceride, a triglyceride, a flavoring agent, a colorant agent, an odorant agent and a therapeutic agent.

139. The thickened oil composition of claim 138, wherein said flavoring agent is selected from the group consisting of sugar, salt, pepper and cumin.

140. The thickened oil composition of claim 119, further comprising at least one therapeutic agent present in an amount of up to 20 times the recommended daily dose per serving unit.

141. The thickened oil composition of claim 140, wherein said at least one therapeutic agent is selected from the group consisting of vitamin A, vitamin C, vitamin D, vitamin E, vitamin B1 (thiamin), vitamin B2 (riboflavin), niacine, nicotinamide, vitamin B6 (pyridoxine), vitamin B12, folic acid, biotin, panthothenic acid, inorganic salts or organometallic complexes of sodium, calcium, iron, iodine, magnesium, zinc, selenium, copper, manganese, chromium and vanadium, powder or extracts of aloe vera, anise, balm, baneberry, basil, thyme, bearberry, bloodroot, cedar, chamomile, chicory, cloves, echinacea, fennel, feverfew, garlic, ginseng, horse nettle, ivy, juniper, lavender, horse nettle, onion, parsley, peppermint, rosemary, sage, saw palmetto berries, St. John's wort, senna, thyme, valerian, yarrow and a mixture thereof.

142. The thickened oil composition of claim 119, further comprising a medicament.

143. A process of producing a thickened oil composition comprising:
(i) blending a major portion of at least one edible oil in combination with at least one edible solidifying agent at a temperature above a melting temperature of said at least one edible solidifying agent, so as to produce the thickened oil composition; and
(ii) cooling said thickened oil composition to ambient temperature.

144. The process of claim 120, further comprising, prior to step (ii):
(iii) adding to said thickened oil composition at least one edible additive selected from the group consisting of a food excipient, a surfactant, an emulsifier, a monoglyceride, a diglyceride, a triglyceride, a flavoring agent, a colorant, an odorant, a herbal substance, a therapeutic agent and a medicament.

145. The process of claim 143, wherein said at least one edible solidifying agent converting said edible oil into a thixotropic composition, whereas, at a temperature above the melting temperature of said at least one edible solidifying agent, said thixotropic composition exhibits a substantially semi-solid state upon rest and a substantially liquid form upon being mechanically disturbed at said temperature.

146. The process of claim 143, wherein said at least one edible solidifying agent is an edible long chain fatty acid and/or an edible solidifying agent having a molecular weight of at least 200 Da and at least one hydroxyl group.

147. The process of claim 143, wherein said at least one edible oil is present at a concentration ranging from 70 to 99 percent by weight and edible solidifying agent is present at a concentration ranging from 1 to 30 percent by weight.

148. The process of claim 146, wherein said edible long chain fatty acid has 18 to 50 carbon atoms in its hydrocarbon chain.

149. The process of claim 146, wherein said edible long chain fatty acid has at least one double bond in its hydrocarbon chain.

150. The process of claim 146, wherein said edible long chain fatty acid has at least one alkyl group side chain.

151. The process of claim 146, wherein said edible solidifying agent having the molecular weight of at least 200 Da and at least one hydroxyl group comprises a fatty alcohol derivative, having at least 15 carbon atoms in its hydrocarbon chain.

152. The process of claim 151, wherein said fatty alcohol derivative has up to about 50 carbon atoms in its hydrocarbon chain.

153. The process of claim 152, wherein said fatty alcohol derivative comprises an edible di-alcohol of formula A: 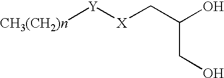
wherein, n=8−48;
X═CHR, O, or NH;
Y═CHR; or C═O; and
R═H or allyl.

154. The process of claim 152, wherein said fatty alcohol derivative comprises an edible di-alcohol of formula B: 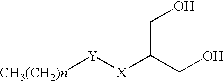
wherein, n=8−48;
X═CHR, O, or NH;
Y═CHR; or C═O; and
R═H or alkyl.

155. The process of claim 143, wherein said at least one edible solidifying agent comprises a wax substance, or a mixture of fatty alcohols derived from a wax substance, a majority of said fatty alcohols having at least 20 carbon atoms in their hydrocarbon chains.

156. The process of claim 143, wherein said at least one edible oil comprises an unsaturated oil.

157. The process of claim 143, wherein said at least one edible oil comprises a major portion of an unsaturated oil.

158. The process of claim 156, wherein said unsaturated oil is present at a concentration range of from 70 to 99.8 percent by weight.

159. The process of claim 156, wherein said unsaturated oil comprises a major portion of poly-unsaturated oils.

160. The process of claim 143, wherein said at least one edible oil includes at least 6 percent of an oil selected from the group consisting of omega-3 oil, omega-6 oil and a mixture thereof.

161. The process of claim 156, wherein said unsaturated oil is of vegetable or marine origin.

162. The process of claim 161, wherein said unsaturated oil is selected from the group consisting of olive oil, soybean oil, canola oil, rapeseed oil, cottonseed oil, coconut oil, palm oil, sesame oil, sunflower oil, borage seed oil, syzigium aromaticum oil, hempseed oil, herring oil, cod-liver oil, salmon oil, corn oil, flaxseed oil, wheat germ oil, evening primrose oil and a mixture thereof.

163. The process of claim 143, wherein said thickened oil composition further comprising at least one edible excipient selected from the group consisting of water, a surfactant, an emulsifier, a diglyceride, a triglyceride, a flavoring agent, a colorant agent, an odorant agent and a therapeutic agent.

164. The process of claim 163, wherein said flavoring agent is selected from the group consisting of sugar, salt, pepper and cumin.

165. The process of claim 143, wherein said thickened oil composition further comprising at least one therapeutic agent present in an amount of up to 20 times the recommended daily dose per serving unit.

166. The process of claim 165, wherein said at least one therapeutic agent is selected from the group consisting of vitamin A, vitamin C, vitamin D, vitamin E, vitamin B 1 (thiamin), vitamin B2 (riboflavin), niacine, nicotinamide, vitamin B6 (pyridoxine), vitamin B12, folic acid, biotin, panthothenic acid, inorganic salts or organometallic complexes of sodium, calcium, iron, iodine, magnesium, zinc, selenium, copper, manganese, chromium and vanadium, powder or extracts of aloe vera, anise, balm, baneberry, basil, thyme, bearberry, bloodroot, cedar, chamomile, chicory, cloves, echinacea, fennel, feverfew, garlic, ginseng, horse nettle, ivy, juniper, lavender, horse nettle, onion, parsley, peppermint, rosemary, sage, saw palmetto berries, St. John's wort, senna, thyme, valerian, yarrow and a mixture thereof.

167. The process of claim 143, wherein said thickened oil composition further comprising a medicament.

168. A food product comprising a base food product or material and a modified fat substitute, wherein said modified fat substitute is a combination of a natural or synthetic fat-based food material and at least one edible solidifying agent, wherein the viscosity or melting temperature of said modified fat substitute is higher than that of said fat-based food material in the absence of said edible solidifying agent.

169. The food product of claim 168, wherein said modified fat substitute is a thixotropic composition, wherein, at a temperature above the melting temperature of said at least one edible solidifying agent, said thixotropic composition exhibits a substantially semi-solid state upon rest and a substantially liquid form upon being mechanically disturbed at said temperature.

170. The food product of claim 168, wherein said at least one edible solidifying agent includes an edible long chain fatty acid and/or an edible solidifying agent having a molecular weight of at least 200 Da and at least one hydroxyl group.

171. The food product of claim 168, wherein said fat-based food material is present at a concentration ranging from 50 to 99 percent by weight.

172. The food product of claim 168, wherein said fat-based food material includes a saturated oil, a mono-unsaturated oil and/or a poly-unsaturated oil.

173. The food product of claim 168, wherein said food product comprises from 0.5 to 99.8 percent by weight of said modified fat substitute.

174. The food product of claim 168, wherein an amount of said at least one edible solidifying agent ranges from 0.1 to 15 percent by weight.

175. The food product of claim 168, wherein said fat-based material comprises at least 50 percent of natural or synthetic unsaturated oils.

176. The food product of claim 171, wherein said fat-based food material is derived from a vegetable, an animal and/or a marine origin.

177. The food product of claim 171, wherein said fat-based food material comprises an edible oil selected from the group consisting of palm oil, palm seed oil, sal oil, mango seed oil, butter oil, cocoa oil, olive oil, corn oil, soybean oil, canola oil, cottonseed oil, coconut oil, sesame oil, sunflower oil, borage seed oil, syzigium aromaticum oil, hempseed oil, herring oil, cod-liver oil, salmon oil, flaxseed oil, wheat germ oil, evening primrose oil, highly saturated oils from animal sources and a mixture thereof.

178. The food product of claim 171, wherein said fat-based food material comprises an oil selected from the group consisting of omega-3, omega-6 and a mixture thereof.

179. The food product of claim 168, wherein said fat-based food material is a margarine, a shortening, a cooking oil or a lard.

180. The food product of claim 170, wherein said edible long chain fatty acid has 18 to 50 carbon atoms in its hydrocarbon chain.

181. The food product of claim 170, wherein said edible long chain fatty acid has at least one double bond in its hydrocarbon chain.

182. The food product of claim 170, wherein said edible long chain fatty acid has at least one alkyl group side chain.

183. The food product of claim 170, wherein said edible solidifying agent having the molecular weight of at least 200 Da and at least one hydroxyl group comprises a fatty alcohol derivative, having at least 15 carbon atoms in its hydrocarbon chain.

184. The food product of claim 183, wherein said fatty alcohol derivative has up to about 50 carbon atoms in its hydrocarbon chain.

185. The food product of claim 184, wherein said fatty alcohol derivative comprises an edible di-alcohol of formula A: 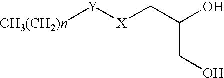
wherein, n=8−48;
X═CHR, O, or NH;
Y═CHR; or C═O; and
R═H or alkyl.

186. The food product of claim 184, wherein said fatty alcohol derivative comprises an edible di-alcohol of formula B: 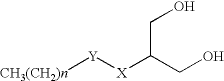
wherein, n=8−48;
X═CHR, O, or NH;
Y═CHR; or C═O; and
R═H or alkyl.

187. The food product of claim 168, wherein said at least one edible solidifying agent comprises a wax substance, or a mixture of fatty alcohols derived from a wax substance, a majority of said fatty alcohols having at least 20 carbon atoms in their hydrocarbon chains.

188. The food product of claim 168, further comprising at least one flavoring agent, coloring agent, odorant agent, therapeutic agent, or combination thereof.

189. The food product of claim 168, further comprising at least one therapeutic agent in an amount up to 20 times the recommended daily dose per serving units.

190. The food product of claim 189, wherein said at least one therapeutic agent is selected from the group consisting of vitamin A, vitamin C, vitamin D, vitamin E, vitamin B 1 (thiamin), vitamin B2 (riboflavin), niacine, nicotinamide, vitamin B6 (pyridoxine), vitamin B12, folic acid, biotin, panthothenic acid, inorganic salts or organometallic complexes of sodium, calcium, iron, iodine, magnesium, zinc, selenium, copper, manganese, chromium and vanadium, powder or extracts of aloe vera, anise, balm, baneberry, basil, thyme, bearberry, bloodroot, cedar, chamomile, chicory, cloves, echinacea, fennel, feverfew, garlic, ginseng, horse nettle, ivy, juniper, lavender, horse nettle, onion, parsley, peppermint, rosemary, sage, saw palmetto berries, St. John's wort, senna, thyme, valerian, yarrow and a mixture there.

191. The food product of claim 168, further comprising a medicament.

192. The food product of claim 168, further comprising an emulsifier system, an admixture of highly saturated triglycerides, or combination thereof.

193. The food product of claim 168, comprising at least 0.5 percent by weight of natural or synthetic unsaturated oils.

194. The food product of claim 168, wherein said base food product or material is selected from the group consisting of a raw fat-based food product, an uncooked fat-based food product and/or a combination thereof.

195. The food product of claim 168, wherein said base food product or material is selected from the group consisting of bread, doughnut, sauce, dressing, coating, spread, salad dressing, margarine, butter, cooking oil, cheese, paste, cake, pastry, muffin, confection, comestible, cream, ice cream, yogurt, frozen yogurt, filling cream and chocolate substitute.

196. The food product of claim 168, wherein said base food product or material is selected from the group consisting of a fresh, processed, or pre-cooked starch, carbohydrate, protein, fat, potato, wheat, corn, barley, oats, rye, millet, vegetable, fruit, tuber, nut, bean, seed, rice, fish, eggs, milk, cheese, meat and poultry.

197. The food product of claim 168, wherein said base food product or material is selected from the group consisting of a fresh, processed, or pre-cooked popcorn, pastry, bread, baked good, pasta, batter, cracker, potato chip, tortilla, snack food, confection and comestible.

198. A process of preparing a modified fat substitute, the process comprising:
(i) selecting a natural or synthetic fat-based food material for which it is desirable to increase the viscosity or melting temperature;
(ii) adding at least one edible solidifying agent to natural or synthetic fat-based material; and
(iii) blending, at the appropriate temperature, so as to produce a modified fat substitute having a higher viscosity or melting temperature than that of said fat-based food material in the absence of said edible solidifying agent.

199. The process of claim 198, further comprising, prior to step (iii):
(iv) adding a desired flavoring, coloring, odorant or therapeutic agent, emulsifier system, admixture of highly saturated triglycerides or combination thereof.

200. The process of claim 198, wherein said modified fat substitute is a thixotropic composition, wherein, at a temperature above the melting temperature of said at least one edible solidifying agent, said thixotropic composition exhibits a substantially semi-solid state upon rest and a substantially liquid form upon being mechanically disturbed at said temperature.

201. The process of claim 198, wherein said at least one edible solidifying agent is an edible long chain fatty acid and/or an edible solidifying agent having a molecular weight of at least 200 Da and at least one hydroxyl group.

202. The process of claim 198, wherein said fat-based food material is present at a concentration ranging from 50 to 99 percent by weight.

203. The process of claim 198, wherein said fat-based food material includes a saturated oil, a mono-unsaturated oil and/or a poly-unsaturated oil.

204. The process of claim 198, wherein said food product comprises from 0.5 to 99.8 percent by weight of said modified fat substitute.

205. The process of claim 198, wherein the at least one edible solidifying agent content in said modified fat substitute ranges from 0.1 to 15 percent.

206. The process of claim 198, wherein said fat-based material comprises at least 50 percent of natural or synthetic unsaturated oils.

207. The process of claim 202, wherein said fat-based food material is derived from a vegetable, an animal and/or a marine origin.

208. The process of claim 202, wherein said fat-based food material comprises an edible oil selected from the group consisting of palm oil, palm seed oil, sal oil, mango seed oil, butter oil, cocoa oil, olive oil, corn oil, soybean oil, canola oil, cottonseed oil, coconut oil, sesame oil, sunflower oil, borage seed oil, syzigium aromaticum oil, hempseed oil, herring oil, cod-liver oil, salmon oil, flaxseed oil, wheat germ oil, evening primrose oil, highly saturated oils from animal sources, and a mixture thereof.

209. The process of claim 202, wherein said fat-based food material comprising an oil selected from the group consisting of omega-3, omega-6 and a mixture thereof.

210. The process of claim 198, wherein said fat-based food material is a margarine, a shortening, a cooking oil or a lard.

211. The process of claim 201, wherein said edible long chain fatty acid has 18 to 50 carbon atoms in its hydrocarbon chain.

212. The process of claim 201, wherein said edible long chain fatty acid has at least one double bond in its hydrocarbon chain.

213. The process of claim 201, wherein said edible long chain fatty acid has at least one alkyl group side chain.

214. The process of claim 201, wherein said edible solidifying agent having the molecular weight of at least 200 Da and at least one hydroxyl group comprises a fatty alcohol derivative, having at least 15 carbon atoms in its hydrocarbon chain.

215. The process of claim 214, wherein said fatty alcohol derivative has up to about 50 carbon atoms in its hydrocarbon chain.

216. The process of claim 215, wherein said fatty alcohol derivative comprises an edible di-alcohol of formula A: 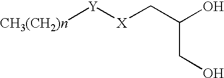
wherein, n=8−48;
X═CHR, O, or NH;
Y═CHR; or C═O; and
R═H or alkyl.

217. The process of claim 215, wherein said fatty alcohol derivative comprises an edible di-alcohol of formula B: 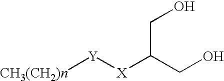
wherein, n=8−48;
X═CHR, O, or NH;
Y═CHR; or C═O; and
R═H or alkyl.

218. The process of claim 198, wherein said at least one edible solidifying agent comprises a wax substance, or a mixture of fatty alcohols derived from a wax substance, a majority of said fatty alcohols having at least 20 carbon atoms in their hydrocarbon chains.

219. The process of claim 198, wherein said modified fat substitute further comprises at least one flavoring agent, coloring agent, odorant agent, therapeutic agent, or combination thereof.

220. The process of claim 198, wherein said modified fat substitute further comprises at least one therapeutic agent in an amount up to 20 times the recommended daily dose per serving units.

221. The process of claim 220, wherein said at least one therapeutic agent is selected from the group consisting of vitamin A, vitamin C, vitamin D, vitamin E, vitamin B1 (thiamin), vitamin B2 (riboflavin), niacine, nicotinamide, vitamin B6 (pyridoxine), vitamin B12, folic acid, biotin, panthothenic acid, inorganic salts or organometallic complexes of sodium, calcium, iron, iodine, magnesium, zinc, selenium, copper, manganese, chromium and vanadium, powder or extracts of aloe vera, anise, balm, baneberry, basil, thyme, bearberry, bloodroot, cedar, chamomile, chicory, cloves, echinacea, fennel, feverfew, garlic, ginseng, horse nettle, ivy, juniper, lavender, horse nettle, onion, parsley, peppermint, rosemary, sage, saw palmetto berries, St. John's wort, senna, thyme, valerian, yarrow and a mixture thereof.

222. The process of claim 198, wherein said modified fat substitute further comprises a medicament.

223. The process of claim 198, wherein said modified fat substitute further comprises an emulsifier system, an admixture of highly saturated triglycerides, or combination thereof.

224. The process of claim 198, wherein said modified fat substitute comprises at least 0.5 percent by weight of natural or synthetic unsaturated oils.

225. A process of preparing a food product, the process comprising:
(i) selecting a natural or synthetic fat-based food material for which it is desirable to increase the viscosity or melting temperature;
(ii) adding at least one edible solidifying agent to said natural or synthetic fat-based material;
(iii) blending, at the appropriate temperature, so as to produce a modified fat substitute having a higher viscosity or melting temperature than that of said fat-based food material in the absence of said edible solidifying agent; and
(iv) applying said fat-based food material onto a base food product or material.

226. The process of claim 225, wherein step (iv) comprises the acts of mixing, blending, pouring, coating, pouring, shaping, inserting, dipping, layering, heating, cooking, boiling, frying, baking, broiling, irradiating or combinations thereof.

227. The process of claim 225, wherein step (iv) comprises the acts of combining with at least one food substance, said food substance is selected from the group consisting of flour, wheat, bran, barley, corn, rice, oats, milk, eggs, fats, oils, vegetable, meat, fish, fruit, nut and combinations thereof.

228. The process of claim 225, wherein step (iv) comprises the acts of combining with at least one food product, substance, or material, said food product, substance or material is selected from the group consisting of a margarine, a butter, a shortening, a cream, a sauce, a dressing, a spread, and combinations thereof.

229. The process of claim 225 wherein step (iv) comprising the acts of combining with at least one food product, said food product is selected from the group consisting of heated, baked, fried, boiled, irradiated, or broiled food product, or combinations thereof.

230. The process of claim 225, further comprising, prior to step (iii):
(v) adding a desired flavoring, coloring, odorant or therapeutic agent, emulsifier system, admixture of highly saturated triglycerides or combination thereof.

231. The process of claim 225, wherein said modified fat substitute is a thixotropic composition, wherein, at a temperature above the melting temperature of said at least one edible solidifying agent, said thixotropic composition exhibits a substantially semi-solid state upon rest and a substantially liquid form upon being mechanically disturbed at said tem.

232. The process of claim 225, wherein said at least one edible solidifying agent is an edible long chain fatty acid and/or an edible solidifying agent having a molecular weight of at least 200 Da and at least one hydroxyl group.

233. The process of claim 225, wherein said fat-based food material is present at a concentration ranging from 50 to 99 percent by weight.

234. The process of claim 225, wherein said fat-based food material includes a saturated oil, a mono-unsaturated oil and/or a poly-unsaturated oil.

235. The process of claim 225, wherein said food product comprises from 0.5 to 99.8 percent by weight of said modified fat substitute.

236. The process of claim 225, wherein the at least one edible solidifying agent content in said modified fat substitute ranges from 0.1 to 15 percent.

237. The process of claim 225, wherein said fat-based material comprises at least 50 percent of natural or synthetic unsaturated oils.

238. The process of claim 233, wherein said fat-based food material is derived from a vegetable, an animal and/or a marine origin.

239. The process of claim 233, wherein said fat-based food material comprising an edible oil selected from the group consisting of palm oil, palm seed oil, sal oil, mango seed oil, butter oil, cocoa oil, olive oil, corn oil, soybean oil, canola oil, cottonseed oil, coconut oil, sesame oil, sunflower oil, borage seed oil, syzigium aromaticum oil, hempseed oil, herring oil, cod-liver oil, salmon oil, flaxseed oil, wheat germ oil, evening primrose oil, highly saturated oils from animal sources and a mixture thereof.

240. The process of claim 233, wherein said fat-based food material comprising an oil selected from the group consisting of omega-3, omega-6 and a mixture thereof.

241. The process of claim 225, wherein said fat-based food material is a margarine, a shortening, a cooking oil or a lard.

242. The process of claim 232, wherein said edible long chain fatty acid has 18 to 50 carbon atoms in its hydrocarbon chain.

243. The process of claim 232, wherein said edible long chain fatty acid has at least one double bond in its hydrocarbon chain.

244. The process of claim 232, wherein said edible long chain fatty acid has at least one alkyl group side chain.

245. The process of claim 232, wherein said edible solidifying agent having the molecular weight of at least 200 Da and at least one hydroxyl group comprises a fatty alcohol derivative, having at least 15 carbon atoms in its hydrocarbon chain.

246. The process of claim 245, wherein said fatty alcohol derivative has up to about 50 carbon atoms in its hydrocarbon chain.

247. The process of claim 246, wherein said fatty alcohol derivative comprises an edible di-alcohol of formula A: 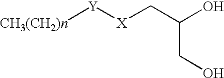
wherein, n=8−48;
X═CHR, O, or NH;
Y═CHR; or C═O; and
R═H or alkyl.

248. The process of claim 246, wherein said fatty alcohol derivative comprises an edible di-alcohol of formula B: 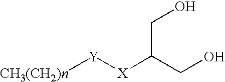
wherein, n=8−48;
X═CHR, O, or NH;
Y═CHR; or C═O; and
R═H or alkyl.

249. The process of claim 225, wherein said at least one edible solidifying agent comprises a wax substance, or a mixture of fatty alcohols derived from a wax substance, a majority of said fatty alcohols having at least 20 carbon atoms in their hydrocarbon chains.

250. The process of claim 225, wherein the food product further comprises at least one flavoring agent, coloring agent, odorant agent, therapeutic agent, or combination thereof.

251. The process of claim 250, wherein said at least one therapeutic agent is in an amount up to 20 times the recommended daily dose per serving units.

252. The process of claim 251, wherein said at least one therapeutic agent is selected from the group consisting of vitamin A, vitamin C, vitamin D, vitamin E, vitamin B1 (thiamin), vitamin B2 (riboflavin), niacine, nicotinamide, vitamin B6 (pyridoxine), vitamin B12, folic acid, biotin, panthothenic acid, inorganic salts or organometallic complexes of sodium, calcium, iron, iodine, magnesium, zinc, selenium, copper, manganese, chromium and vanadium, powder or extracts of aloe vera, anise, balm, baneberry, basil, thyme, bearberry, bloodroot, cedar, chamomile, chicory, cloves, echinacea, fennel, feverfew, garlic, ginseng, horse nettle, ivy, juniper, lavender, horse nettle, onion, parsley, peppermint, rosemary, sage, saw palmetto berries, St. John's wort, senna, thyme, valerian, yarrow and a mixture thereof.

253. The process of claim 225, wherein said food product further comprises a medicament.

254. The process of claim 225, wherein said food product further comprises an emulsifier system, an admixture of highly saturated triglycerides, or a combination thereof.

255. The process of claim 225, wherein said food product comprises at least 0.5 percent by weight of natural or synthetic unsaturated oils.

256. The process of claim 225, wherein said base food product or material is selected from the group consisting of a raw fat-based food product, an uncooked fat-based food product and/or a combination thereof.

257. The process of claim 225, wherein said base food product or material is selected from the group consisting of bread, doughnut, sauce, dressing, coating, spread, salad dressing, margarine, butter, cooking oil, cheese, paste, cake, pastry, muffin, confection, comestible, cream, ice cream, yogurt, frozen yogurt, filling cream, and chocolate substitute.

258. The process of claim 225, wherein said base food product or material is selected from the group consisting of a fresh, processed, or pre-cooked starch, carbohydrate, protein, fat, potato, wheat, corn, barley, oats, rye, millet, vegetable, fruit, tuber, nut, bean, seed, rice, fish, eggs, milk, cheese, meat and poultry.

259. The process of claim 225, wherein said base food product or material is selected from the group consisting of a fresh, processed, or pre-cooked popcorn, pastry, bread, baked good, pasta, batter, cracker, potato chip, tortilla, snack food, confection and comestible.

260. An edible paste comprising a combination of at least one therapeutic edible oil and at least one edible solidifying agent, said edible solidifying agent being for converting said edible oil into said edible paste.

261. The edible paste of claim 260, wherein said edible paste is a thixotropic composition, said at least one edible solidifying agent converting said edible oil into said thixotropic composition, wherein said thixotropic composition exhibits a substantially semi-solid state upon rest at a temperature above the melting temperature of said at least one edible solidifying agent and a substantially liquid form upon being mechanically disturbed at said temperature.

262. The edible paste of claims 260, wherein said at least one edible solidifying agent includes an edible long chain fatty acid and/or an edible solidifying agent having a molecular weight of at least 200 Da and at least one hydroxyl group.

263. The edible paste of claim 260, wherein said at least one edible oil is present at a concentration ranging from 50 to 99 percent by weight of said edible paste.

264. The edible paste of claim 262, wherein said edible long chain fatty acid has 18 to 50 carbon atoms in its hydrocarbon chain.

265. The edible paste of claim 262, wherein said edible long chain fatty acid has at least one double bond in its hydrocarbon chain.

266. The edible paste of claim 262, wherein said edible long chain fatty acid has at least one branched alkyl group side chain.

267. The edible paste of claim 260, wherein said at least one edible oil comprises an unsaturated oil.

268. The edible paste of claim 260, wherein said at least one edible oil comprises a major portion of an unsaturated oil.

269. The edible paste of claim 267, wherein said unsaturated oil is present at a concentration range of from 70 to 99.8 percent by weight.

270. The edible paste of claim 267, wherein said unsaturated oil comprises a major portion of a poly-unsaturated oil.

271. The edible paste of claim 260, wherein said at least one edible oil includes at least 6 percent by weight of an oil selected from the group consisting of omega-3, omega-6 oil and a mixture thereof.

272. The edible paste of claim 260, wherein at least one edible oil is derived from a vegetable, a marine origin or a combination thereof.

273. The edible paste of claim 260, wherein said at least one edible oil is selected from the group consisting of evening primrose oil, castor oil, syzigium aromaticuin oil, linen oil, matthiola incana oil, flaxseed oil, hempseed oil, garlic oil, herring oil, salmon oil, cod liver oil, shark oil, sunflower oil, olive oil, hypericum oil, borage seed oil and sweet basil oil.

274. The edible paste of claim 260, further comprising at least one edible excipient, selected from the group consisting of water, a surfactant, an emulsifier, a monoglyceride, a diglyceride, a triglyceride, a flavoring agent, a colorant agent and an odorant agent.

275. The edible paste of claim 274, wherein said flavoring agent is selected from the group consisting of sugar, salt, pepper and cumin.

276. The edible paste of claim 260, further comprising at least one therapeutic agent in an amount up to 20 times the recommended daily dose per serving unit.

277. The edible paste of claim 276, wherein said at least one therapeutic agent is selected from the group consisting of vitamin A, vitamin C, vitamin D, vitamin E, vitamin B 1 (thiamin), vitamin B2 (riboflavin), niacine, nicotinamide, vitamin B6 (pyridoxine), vitamin B12, folic acid, biotin, panthothenic acid, inorganic salts or organometallic complexes of sodium, calcium, iron, iodine, magnesium, zinc, selenium, copper, manganese, chromium and vanadium, powder or extracts of aloe vera, anise, balm, baneberry, basil, thyme, bearberry, bloodroot, cedar, chamomile, chicory, cloves, echinacea, fennel, feverfew, garlic, ginseng, horse nettle, ivy, juniper, lavender, horse nettle, onion, parsley, peppermint, rosemary, sage, saw palmetto berries, St. John's wort, senna, thyme, valerian, yarrow and a mixture thereof.

278. The edible paste of claim 262, wherein said edible solidifying agent having the molecular weight of at least 200 Da and at least one hydroxyl group comprises a fatty alcohol derivative having at least 15 carbon atoms in its hydrocarbon chain.

279. The edible paste of claim 278, wherein said fatty alcohol derivative has up to about 50 carbon atoms in its hydrocarbon chain.

280. The edible paste of claim 279, wherein said fatty alcohol derivative comprises an edible di-alcohol of formula A: 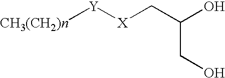
wherein, n=8−48;
X═CHR, O, or NH;
Y═CHR; or C═O; and
R═H or alkyl.

281. The edible paste of claim 279, wherein said fatty alcohol derivative comprises an edible di-alcohol of formula B: 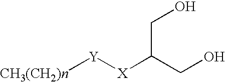
wherein, n=8−48;
X═CHR, O, or NH;
Y═CHR; or C═O; and
R═H or alkyl.

282. The edible paste of claim 260, further comprising a medicament.

283. The edible paste of claim 260, wherein said at least one solidifying agent comprises a wax substance, or a mixture of fatty alcohols derived from a wax substance, a majority of said fatty alcohols having at least 20 carbon atoms in their hydrocarbon chains.

284. The edible paste of claim 260, wherein said at least one edible solidifying agent is a component of a natural product.

285. The edible paste of claim 260, comprising at least 90 percent by weight of a therapeutic oil and a mixture of fatty alcohols derived from a wax substance, a majority of said fatty alcohols having at least 20 carbon atoms in their hydrocarbon chains.

286. The edible paste according to claim 260, comprising unsaturated oil having at least 2 double bonds.

287. The edible paste of claim 260, wherein said therapeutic oil is canola oil.

288. The edible paste of claim 260, wherein said therapeutic oil is a marine oil in combination with flavoring additives.

289. The edible paste of claim 260, wherein said therapeutic oil is primrose oil.

290. The edible paste of claim 260, further comprising a cocoa powder.

291. The edible paste of claim 260, wherein said therapeutic oil is selected from an edible mineral oil, an edible silicone oil and a mixture thereof.

292. The edible paste of claim 260, comprising at least 95 percent by weight of said therapeutic oil therein.

293. The edible paste of claim 260, comprising at least 99 percent by weight of said therapeutic oil therein.

294. The edible paste of claim 260, wherein said edible paste includes cetyl alcohol and the weight percentage of said cetyl alcohol in said edible paste is less than about 2 percent.

295. The edible paste of claim 260, wherein said edible paste includes stearyl alcohol and the weight percentage of said stearyl alcohol in said edible paste is less than about 2 percent.

296. The edible paste of claim 260, further comprising solid components having a specific gravity of between 0.6 and 1.4 wherein said solid components are evenly distributed in the edible paste.

297. The edible paste of claim 260, for use as a lipid lowering remedy, comprising:
(a) 80 to 99.8 percent by weight of an omega 3 oil; and
(b) at least one edible solidifying agent, said edible solidifying agent comprising at least one fatty alcohol derivative having at least 15 carbon atoms in its hydrocarbon chain.

298. The edible paste of claim 260, for use as a diabetes remedy, comprising:
(a) 80 to 99.8 percent by weight of an omega 3 oil;
(b) at least one edible solidifying agent, said edible solidifying agent comprising at least one fatty alcohol derivative having at least 15 carbon atoms in its hydrocarbon chain; and
(c) up to 1 percent by weight of at least one trace mineral, selected from vanadium, chromium, tungsten, zinc and a mixture thereof.

299. The edible paste of claim 260, for use as a constipation remedy, comprising:
(a) 80 to 99.8 percent by weight of an omega 3 oil; and
(b) at least one edible solidifying agent, said edible solidifying agent comprising at least one fatty alcohol derivative having at least 15 carbon atoms in its hydrocarbon chain; and

300. The edible paste of claim 260, for use as a hyperactivity remedy, comprising:
(a) 80 to 99.8 percent by weight of an omega 3 oil; and
(b) at least one edible solidifying agent, said edible solidifying agent comprising at least one fatty alcohol derivative having at least 15 carbon atoms in its hydrocarbon chain.

301. A method of consuming therapeutically effective amounts of a therapeutic oil, the method comprising consuming an edible paste including a combination of at least one therapeutic edible oil and at least one edible solidifying agent, said edible solidifying agent being for converting said edible oil into said edible paste.

302. The method of claim 301, wherein said edible paste is a thixotropic composition, said at least one edible solidifying agent converting said edible oil into said thixotropic composition, wherein said thixotropic composition exhibits a substantially semi-solid state upon rest at a temperature above the melting temperature of said at least one edible solidifying agent and a substantially liquid form upon being mechanically disturbed at said temperature.

303. The method of claims 301, wherein said at least one edible solidifying agent includes an edible long chain fatty acid and/or an edible solidifying agent having a molecular weight of at least 200 Da and at least one hydroxyl group.

304. The method of claim 301, wherein said at least one edible oil is present at a concentration ranging from 50 to 99 percent by weight of said edible paste.

305. The method of claim 303, wherein said edible long chain fatty acid has 18 to 50 carbon atoms in its hydrocarbon chain.

306. The method of claim 303, wherein said edible long chain fatty acid has at least one double bond in its hydrocarbon chain.

307. The method of claim 303, wherein said edible long chain fatty acid has at least one branched alkyl group side chain.

308. The method of claim 301, wherein said at least one edible oil comprises an unsaturated oil.

309. The method of claim 301, wherein said at least one edible oil comprises a major portion of an unsaturated oil.

310. The method of claim 308, wherein said unsaturated oil is present at a concentration range of from 70 to 99.8 percent by weight.

311. The method of claim 308, wherein said unsaturated oil comprises a major portion of a poly-unsaturated oil.

312. The method of claim 301, wherein said at least one edible oil includes at least 6 percent by weight of an oil selected from the group consisting of omega-3 oil, omega-6 oil and a mixture thereof.

313. The method of claim 301, wherein said at least one edible oil is derived from a vegetable, a marine origin or a combination thereof.

314. The method of claim 301, wherein said at least one edible oil is selected from the group consisting of evening primrose oil, castor oil, syzigium aromaticum oil, linen oil, matthiola incana oil, flaxseed oil, hempseed oil, garlic oil, herring oil, salmon oil, cod liver oil, shark oil, sunflower oil, olive oil, hypericum oil, borage seed oil and sweet basil oil.

315. The method of claim 301, wherein said edible paste further comprises at least one edible excipient, selected from the group consisting of water, a surfactant, an emulsifier, a monoglyceride, a diglyceride, a triglyceride, a flavoring agent, a colorant agent and an odorant agent.

316. The method of claim 315, wherein said flavoring agent is selected from the group consisting of sugar, salt, pepper and cumin.

317. The method of claim 301, wherein said edible paste further comprises at least one therapeutic agent in an amount up to 20 times the recommended daily dose per serving unit.

318. The method of claim 317, wherein said at least one therapeutic agent is selected from the group consisting of vitamin A, vitamin C, vitamin D, vitamin E, vitamin B1 (thiamin), vitamin B2 (riboflavin), niacine, nicotinamide, vitamin B6 (pyridoxine), vitamin B12, folic acid, biotin, panthothenic acid, inorganic salts or organometallic complexes of sodium, calcium, iron, iodine, magnesium, zinc, selenium, copper, manganese, chromium and vanadium, powder or extracts of aloe vera, anise, balm, banebeny, basil, thyme, bearberry, bloodroot, cedar, chamomile, chicory, cloves, echinacea, fennel, feverfew, garlic, ginseng, horse nettle, ivy, juniper, lavender, horse nettle, onion, parsley, peppermint, rosemary, sage, saw palmetto berries, St. John's wort, senna, thyme, valerian, yarrow and a mixture thereof.

319. The method of claim 303, wherein said edible solidifying agent having the molecular weight of at least 200 Da and at least one hydroxyl group comprises a fatty alcohol derivative having at least 15 carbon atoms in its hydrocarbon chain.

320. The method of claim 319, wherein said fatty alcohol derivative has up to about 50 carbon atoms in its hydrocarbon chain.

321. The method of claim 320, wherein said fatty alcohol derivative comprises an edible di-alcohol of formula A: 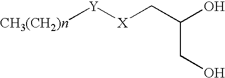
wherein, n=8−48;
X═CHR, O, or NH;
Y═CHR; or C═O; and
R═H or alkyl.

322. The method of claim 320, wherein said fatty alcohol derivative comprises an edible di-alcohol of formula B: 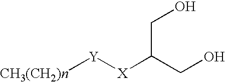
wherein, n=8−48;
X═CHR, O, or NH;
Y═CHR; or C═O; and
R═H or allyl.

323. The method of claim 301, wherein said edible paste further comprises a medicament.

324. The method of claim 301, wherein said at least one solidifying agent comprises a wax substance, or a mixture of fatty alcohols derived from a wax substance, a majority of said fatty alcohols having at least 20 carbon atoms in their hydrocarbon chains.

325. The use of claim 301, wherein said at least one edible solidifying agent is a component of a natural product.

326. A method of preparing an edible paste, comprising mixing at least one therapeutic edible oil and at least one edible solidifying agent, said edible solidifying agent being for converting said edible oil into said edible paste.

327. The method of claim 327, wherein said edible paste is a thixotropic composition, said at least one edible solidifying agent converting said edible oil into said thixotropic composition, wherein said thixotropic composition exhibits a substantially semi-solid state upon rest at a temperature above the melting temperature of said at least one edible solidifying agent and a substantially liquid form upon being mechanically disturbed at said temperature.

328. The method of claims 327, wherein said at least one edible solidifying agent includes an edible long chain fatty acid and/or an edible solidifying agent having a molecular weight of at least 200 Da and at least one hydroxyl group.

329. The method of claim 327, wherein said at least one edible oil is present at a concentration ranging from 50 to 99 percent by weight of said edible paste.

330. The method of claim 328, wherein said edible long chain fatty acid has 18 to 50 carbon atoms in its hydrocarbon chain.

331. The method of claim 328, wherein said edible long chain fatty acid has at least one double bond in its hydrocarbon chain.

332. The method of claim 328, wherein said edible long chain fatty acid has at least one branched alkyl group side chain.

333. The method of claim 327, wherein said at least one edible oil comprises an unsaturated oil.

334. The method of claim 327, wherein said at least one edible oil comprises a major portion of an unsaturated oil.

335. The method of claim 333, wherein said unsaturated oil is present at a concentration range of from 70 to 99.8 percent by weight.

336. The method of claim 333, wherein said unsaturated oil comprises a major portion of a poly-unsaturated oil.

337. The method of claim 327, wherein said at least one edible oil includes at least 6 percent by weight of an oil selected from the group consisting of omega-3 oil, omega-6 oil and a mixture thereof.

338. The method of claim 327, wherein said at least one edible oil is derived from a vegetable, a marine origin or a combination thereof.

339. The method of claim 327, wherein said at least one edible oil is selected from the group consisting of evening primrose oil, castor oil, syzigium aromaticum oil, linen oil, matthiola incana oil, flaxseed oil, hempseed oil, garlic oil, herring oil, salmon oil, cod liver oil, shark oil, sunflower oil, olive oil, hypericum oil, borage seed oil and sweet basil oil.

340. The method of claim 327, further comprising adding into said edible paste at least one edible excipient selected from the group consisting of water, a surfactant, an emulsifier, a monoglyceride, a diglyceride, a triglyceride, a flavoring agent, a colorant agent and an odorant agent.

341. The method of claim 340, wherein said flavoring agent is selected from the group consisting of sugar, salt, pepper and cumin.

342. The method of claim 327, further comprising adding to said edible paste at least one therapeutic agent in an amount up to 20 times the recommended daily dose per serving unit.

343. The method of claim 342, wherein said at least one therapeutic agent is selected from the group consisting of vitamin A, vitamin C, vitamin D, vitamin E, vitamin B1 (thiamin), vitamin B2 (riboflavin), niacine, nicotinamide, vitamin B6 (pyridoxine), vitamin B12, folic acid, biotin, panthothenic acid, inorganic salts or organometallic complexes of sodium, calcium, iron, iodine, magnesium, zinc, selenium, copper, manganese, chromium and vanadium, powder or extracts of aloe vera, anise, balm, baneberry, basil, thyme, bearberry, bloodroot, cedar, chamomile, chicory, cloves, echinacea, fennel, feverfew, garlic, ginseng, horse nettle, ivy, juniper, lavender, horse nettle, onion, parsley, peppermint, rosemary, sage, saw palmetto bernies, St. John's wort, senna, thyme, valerian, yarrow and a mixture thereof.

344. The method of claim 328, wherein said edible solidifying agent having the molecular weight of at least 200 Da and at least one hydroxyl group comprises a fatty alcohol derivative having at least 15 carbon atoms in its hydrocarbon chain.

345. The method of claim 344, wherein said fatty alcohol derivative has up to about 50 carbon atoms in its hydrocarbon chain.

346. The method of claim 345, wherein said fatty alcohol derivative comprises an edible di-alcohol of formula A: 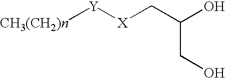
wherein, n=8−48;
X═CHR, O, or NH;
Y═CHR; or C═O; and
R═H or allyl.

347. The method of claim 345, wherein said fatty alcohol derivative comprises an edible di-alcohol of B: 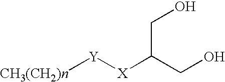
wherein, n=8−48;
X═CHR, O, or NH;
Y═CHR; or C═O; and
R═H or alkyl.

348. The method of claim 327, further comprising adding to said edible paste a medicament.

349. The method of claim 327, wherein said at least one solidifying agent comprises a wax substance, or a mixture of fatty alcohols derived from a wax substance, a majority of said fatty alcohols having at least 20 carbon atoms in their hydrocarbon chains.

350. The method of claim 327, wherein said at least one edible solidifying agent is a component of a natural product.

351. The method of claim 327, wherein said edible paste comprising at least 90 percent by weight of a therapeutic oil and a mixture of fatty alcohols derived from a wax substance, a majority of said fatty alcohols having at least 20 carbon atoms in their hydrocarbon chains.

352. The edible paste according to claim 327, wherein said edible paste comprising unsaturated oil having at least 2 double bonds.

353. The method of claim 327, wherein said therapeutic oil is canola oil.

354. The method of claim 327, wherein said therapeutic oil is a marine oil in combination with flavoring additives.

355. The method of claim 327, wherein said therapeutic oil is primrose oil.

356. The method of claim 327, wherein said edible paste further comprising a cocoa powder.

357. The method of claim 327, wherein said therapeutic oil is selected from an edible mineral oil, an edible silicone oil and a mixture thereof.

358. The method of claim 327, wherein said edible paste comprises at least 95 percent by weight of said therapeutic oil therein.

359. The method of claim 327, wherein said edible paste comprises at least 99 percent by weight of said therapeutic oil therein.

360. The method of claim 327, wherein said edible paste includes cetyl alcohol and the weight percentage of said cetyl alcohol in said edible paste is less than about 2 percent.

361. The method of claim 327, wherein said edible paste includes stearyl alcohol and the weight percentage of said stearyl alcohol in said edible paste is less than about 2 percent.

362. The method of claim 327, wherein said edible paste further comprises solid components having a specific gravity of between 0.6 and 1.4 wherein said solid components are evenly distributed in the edible paste.

363. A liquid dairy product comprising a homogeneous mixture of substantially fat-free milk or milk powder dissolved in water and an edible thickened oil composition, said edible thickened oil composition including a combination of at least one edible oil and at least one edible solidifying agent, said edible solidifying agent being for converting said edible oil into said thickened oil composition, the liquid dairy product having a consistency and taste of fat milk.

364. A solidified dairy product comprising a homogeneous mixture of substantially fat-free milk or milk powder dissolved in water and an edible thickened oil composition, said edible thickened oil composition including a combination of at least one edible oil and at least one edible solidifying agent, said edible solidifying agent being for converting said edible oil into said thickened oil composition, the solidified dairy product having a consistency and taste of solidified fat cheese.

365. The solidified dairy product of claim 364, having thixotropic properties and is therefore readily cut-through with a blade.

366. A semi-solid or solid dairy product comprising a homogeneous mixture of substantially fat-free milk or milk powder dissolved in water and an edible thickened oil composition, said edible thickened oil composition including a combination of at least one edible oil and at least one edible solidifying agent, said edible solidifying agent being for converting said edible oil into said thickened oil composition, the semi-solid or solid dairy product having a consistency of a conventional semi-solid or solid dairy product.
